SEQUENCE LISTING (2 sequences)

SEQ ID NO: 1 - protein, 12 aa, Artificial Sequence
  Synthetically derived  peptide of BRCA2
TFVSPAAQKAGG

SEQ ID NO: 2 - protein, 11 aa, Artificial Sequence
  Synthetically derived  peptide of BRCA2
TFVSPAAKAGG

(19) United States
(12) Patent Application Publication
(10) Pub. No.: US 2010/0035281 A1
(43) Pub. Date: Feb. 11, 2010

(54) COMPOSITIONS AND METHODS FOR TISSUE-BASED PROTEIN TRUNCATION TEST FOR DISEASE DIAGNOSIS

(76) Inventor: Jeffrey T. Holt, Aurora, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/442,771

(22) PCT Filed: Sep. 25, 2007
(86) PCT No.: PCT/US07/79470

§ 371 Date: Mar. 25, 2009

Related U.S. Application Data
(60) Provisional application No. 60/847,218, filed on Sep. 25, 2006.

(51) Int. Cl. G01N 33/574; G01N 33/53; C07K 16/00

(52) U.S. Cl. 435/7.23; 435/7.21; 530/387.1

(57) ABSTRACT

Embodiments herein concern methods and compositions for diagnosing or predicting the onset of a genetic disorder In certain embodiments, the methods may include use of a rapid and inexpensive assay. In other embodiments, the assay utilizes a tissue sample from a subject to identify the state of a target protein known to associate with a genetic disorder In accordance with these embodiments, the target protein may have a DNA sequence change such as a mutation, deletion, insertion or substitution leading to generation of a termination codon and truncation of the protein. In some embodiments, a composition of an antibody directed to bind a protein associated with a disorder may be used to predict onset of the disorder or predict sensitivity of the disorder to therapeutic treatments.

### REFERENCE TO RELATED APPLICATIONS

This application claims benefit of provisional U.S. Application No. 60/847,218, filed Sep. 25, 2006 and is hereby incorporated herein by reference in its entirety for all purposes.

### FIELD

The present invention relates to methods and compositions for assessing presence or risk of a disorder in a subject. In some embodiments, the present invention relates to diagnosing or predicting the onset of a genetic disorder and/or therapeutic treatment for the disorder based on the level of post-translational modification alteration in a target protein known to be associated with the disorder. In another embodiment, methods for diagnosing or predicting the onset of a genetic disorder are based on the level of truncation of a target protein due to a change in the DNA sequence of the target protein.

### BACKGROUND

An increasing number of genes that play a role in many different disorders are being identified. In certain disorders, detection of mutations in these genes is instrumental in determining susceptibility to or diagnosing the presence of a disorder. Some disorders, such as certain breast or prostate cancers, are caused by a single or very few mutations present in a gene. In such cases where one or only a few known mutations are responsible for the disorder, methods for detecting the mutations are targeted to the site within the gene at which they are known to occur. However, the mutation responsible for such a disorder can only be established if there exists an accurate method for identifying the mutation.

In many other cases individuals affected by a given disease display extensive allelic heterogeneity. For example, more than 125 mutations in the human BRCA1 gene have been reported. Mutations in the BRCA1 gene are thought to account for roughly 45% of inherited breast cancer and 80-90% of families with increased risk of early onset breast and ovarian cancer.

Breast cancer is also an example of a disease which has genetic heterogeneity. In addition to BRCA1, the BRCA2 and BRCA3 genes have been linked to breast cancer. Similarly, the NFI and NFII genes are involved in neurofibromatosis (types I and II, respectively). Furthermore, hereditary non-polyposis colorectal cancer (HNPCC) is a disease in which four genes, MSH2, MLH1, PMS1, and PMS2, have been implicated.

Rapid identification of a genetic mutation or genetic alteration can provide for early diagnosis which is essential for effective treatment of many disorders. In the current state of the art in the diagnosis and treatment of disorders such as inherited or sporadic disorders, for example cancers and blood disorder, it is of critical importance that a clinician or pathologist be able to diagnose the condition early in the stage of disease in a patient. The earlier the clinician or pathologist can detect a tumor, blood disorder, or other disorder, the greater the chances that an efficacious, minimally toxic treatment can be devised. At the present time, however, there are few rapid and reliable diagnostic methods in clinical use for diagnosing various types of genetic disorders or risk of developing a genetic disorder, particularly those having a high degree of prevalence. In addition, understanding the mechanisms responsible for hereditary cancers can lead to an impact on the diagnosis and treatment of more common cancers such as sporadic cancers.

Despite efforts to fight cancer, many malignant diseases that are of interest continue to present major challenges to clinical oncology. For example, prostate cancer is the second most common cause of cancer deaths in men. Current treatment protocols such as cytotoxic chemotherapy have been disappointing, indicating a long felt need for new approaches to treatment of advanced prostatic cancer. Other diseases resulting from abnormal cell replication, for example metastatic melanomas, brain tumors of glial origin (e.g., astrocytomas), and lung adenocarcinoma, are also highly aggressive malignancies with poor prognosis especially if diagnosed at advanced stages. The incidence of breast cancer and lung adenocarcinoma has been increasing significantly in recent years. Surgical treatments of brain tumors often fail to remove all tumor tissues, resulting in recurrences. Systemic chemotherapy is hindered by blood barriers. Therefore, there is an urgent need for new early detection, rapid and inexpensive approaches to diagnosis of recessive genetic or dominantly inherited genetic disorders, including but not limited to prostatic cancer, melanoma, breast cancer, skin cancer, blood disorders.

Several other markers specific for particular tumor or cancer types have been used with increasing frequency over the last five years. BRCA1 and BRCA2 in the context of breast and ovarian cancer are examples of such markers.

Despite many scientific advances in recent years to better predict disorders on the level of gene mutations, such disorders continue to cause long-term disability in a significant number of patients. The ability to predict the potential onset or predisposition to a disorder is an important goal for patients and their treating clinicians in order to maximize the potential for early intervention and monitoring of the patient.

A need still exists for an inexpensive and rapid assays that detect a change in post translational modification of proteins or mutations that cause a truncation in a protein. Such an assay would allow early evaluation of an individual having or at risk for development of a target disorder and potential treatment.

### SUMMARY

Embodiments of the present invention relate to methods and compositions for early detection of, or predisposition for, a genetic disorder in a subject. In accordance with these embodiments, one or more tissue samples may be obtained from a subject and a target protein of the sample(s) can be analyzed for binding by one or more antibodies including but not limited to carboxy-terminal antibodies, amino-terminal antibodies, or phosphospecific antibodies.

In certain embodiments, early detection of a genetic disorder in a subject may include early detection of a recessive genetic disorder or a dominantly inherited genetic disorder in a subject not previously diagnosed with the disorder. (see Appendix B, incorporated herein by reference in its entirety and for all purposes) In accordance with these embodiments, one or more tissue samples of a subject for analysis may include, but are not limited to breast, prostate, ovarian, pancreatic, lung, brain, thyroid, bowel, skin and throat tissue samples. These samples may be used to assess the state or alteration of one or more target proteins present in the tissue sample. In one example, a target protein of a tissue sample may be targeted with a carboxy-terminal directed antibody to assess the level of binding of the antibody. In another example, a target protein of a tissue sample may be targeted with a carboxy-terminal directed antibody and an amino-terminal directed antibody to assess the ratio of carboxy-terminal directed antibody binding levels to amino-terminal directed antibody binding levels. In accordance with these embodiments, the level of binding of the antibody or the ratio of binding between the different antibodies may be used to assess the risk of developing a disorder or detecting the presence of a previously undiagnosed disorder in the subject.

In some embodiments of the present invention, a disorder can be an inherited disorder which can include, but is not limited to, a storage disease, urea cycle disorders, endocrine disorders, mitochondrial disease, lysosomal disease, or secretory disorders. In other embodiments, disorders contemplated herein can include disorders of carbohydrate metabolism (e.g., glycogen storage disease), disorders of amino acid metabolism (e.g. phenylketonuria), maple syrup urine disease, glutaric acidemia type 1, disorders of organic acid metabolism (e.g. organic acidurias), disorders of fatty acid oxidation and mitochondrial metabolism (e.g., medium chain acyl dehydrogenase deficiency (glutaric acidemia type 2), disorders of porphyrin metabolism (e.g., acute intermittent porphyria), disorders of purine or pyrimidine metabolism (e.g., Lesch-Nyhan syndrome), disorders of steroid metabolism (e.g., congenital adrenal hyperplasia), disorders of mitochondrial function (e.g., Kearns-Sayre syndrome), disorders of peroxisomal function (e.g., Zellweger syndrome), or lysosomal storage disorders (e.g., Gaucher's disease).

In other embodiments of the present invention, responsiveness to a therapeutic agent of a genetic disorder may be assessed. In accordance with these embodiments, a subject having or suspected of developing a genetic disorder may be assessed for responsiveness to a potential therapeutic treatment. The responsiveness of a disorder to a therapeutic treatment may be assessed using a phosphospecific antibody. A phosphospecific antibody as used herein may be directed to bind one or more phosphorylated amino acids of a target protein. In accordance with these embodiments, one or more tissue samples of the same or different origins may be used to detect the level of binding of one or more phosphospecific antibodies to the target proteins of the samples. In one particular example, a target protein of a tissue sample may be exposed to a phosphospecific antibody such as any phosphospecific antibody composition as contemplated herein. One particular example of a phophospecific antibody may include a composition of the present invention, serine 3291BRCA2 phosphospecific antibody. In accordance with this example, the level of binding of the serine 3291BRCA2 phosphospecific antibody may be analyzed and the bound antibody may be correlated with potential responsiveness to a therapeutic agent such as a chemotherapeutic agent. Example therapeutic treatments include, but are not limited to, targeting BRCA2 function using a therapeutic agent, using homologous recombination therapy, using radiation therapy, or using drugs that inhibit DNA repair. Examples of therapeutic agents include, but are not limited to, homologous recombination therapy examples: mitomycin C, PARP inhibitors including 3-amino-benzamide, 8-hydroxy-2-methylquinazolin-4-(3H)-one (NU1025), AG14361 (see Table 4 for more examples); radiation therapy examples: including direct beam radiation, implanted source radiation, focused or refined beam radiation; agent inhibiting DNA repair including: doxorubicine, cycophosphamide, actinomycin D, bleomycin, irinotecan, and cis-platinum.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIGS. 1A-1D represent exemplary immunohistochemistry (IHC) analyses using carboxy-terminal antibodies.

FIGS. 2A-2F represent exemplary immunohistochemistry (IHC) analyses using carboxy-terminal antibodies.

FIGS. 3A-3C represent exemplary immunohistochemistry (IHC) analyses using carboxy-terminal antibodies (A), amino-terminal antibodies (B) and IgG as a negative control (C).

FIGS. 4A-4C represents an exemplary Western blot using a phosphospecific antibody.

FIGS. 5A-5C represent exemplary Western blots exposed to various antibodies and agents.

FIGS. 6A-6C represent exemplary immunohistograms (IHC) of tissue samples after exposure to various antibodies and agents

FIG. 7 represents an exemplary schematic of drug sensitivity and presence or absence of phosphorylation.

FIGS. 8A-8D represent exemplary IHCs performed on human breast tissue using a C-terminal specific monoclonal antibody.

FIGS. 9A-9C represent exemplary IHCs performed on MCF7 cells using a phosphorylation-specific monoclonal antibody.

FIGS. 10A-10C represent exemplary IHCs performed on human breast tissue using a phosphorylation-specific monoclonal antibody.

FIGS. 11A-11F represent exemplary IHCs performed on skeletal muscle using C-terminal-specific antibodies.

### DESCRIPTION OF ILLUSTRATIVE EMBODIMENTS

### Definitions

As used herein, “a” or “an” may mean one or more than one of an item.

As used herein, “modulation” refers to a change in the level or magnitude of an activity or process. The change may be either an increase or a decrease. For example, modulation may refer to either an increase or a decrease in activity or levels. Modulation may be assayed by determining any parameter that indirectly or directly affects or reflects truncation of a protein or a change in post-translational modification of a protein.

### DETAILED DESCRIPTION

In the following section, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods or components have not been included in the description.

Embodiments of the present invention relate to methods and compositions for early detection of, or predisposition for, a genetic disorder in a subject.

Healthcare providers are in need of an inexpensive, rapid and easy methods to detect genetic alteration(s) of target proteins known to confer predisposition to a genetic disorder. Because of the nature of genetic disorders, for example cancer or inherited diseases (e.g. metabolic diseases) as a dynamic on-going process, a method that can rapidly track a mutational change would be beneficial. In certain embodiments, identification of mutational changes in a target gene can be important for a subject having, or with a propensity for, a recessive genetic disorder or a dominantly inherited genetic disease like cancer.

In some embodiments, a health care provider can obtain a tissue sample from a subject having or with a propensity for a recessive genetic disorder or a dominantly inherited genetic disease (see Table 4). In accordance with these embodiments, the sample can be tested for one or more mutations in a target gene in order to assess whether the subject is pre-disposed to a particular illness. Tissue samples contemplated in the present invention may include but are not limited to breast, prostate, ovarian, pancreatic, lung, brain, thyroid, bowel, skin, gastrointestinal, buccal and throat tissue samples. In addition, embodiments disclosed herein are directed to early detection of genetic disorders whose onset is directly linked to an alteration in one or more genes leading to an alteration in the translated protein of the gene(s), such as a mutation causing a change in a post translational modifications (e.g. phosphorylation, methylation, maturation of the protein via cleavage, sulfation, Cysteine/Methionine oxidation, N-acetylation, lipidation, proteolysis, ubiquitylation, glycosylation, ADP-ribosylation, hydroxylation, automodification, carboxylation, and modification with biotin, lipoate and phosphopantetheine) of a protein or truncation of a protein. Other situations where these techniques may be useful may include early diagnosis of onset or prediction of propensity for getting a genetic disorder such as those disclosed in Table 4. In certain particular examples genes associated with disorders considered herein include, but are not limited to BRCA2, BRCA1, Familial Polyposis (FAP), Duchenne's Muscular Dystrophy, and Beta-thalassemia.

Some embodiments of the present invention relate to methods to analyze a tissue sample in order to detect truncation of a target protein. These methods can include a simple, inexpensive and rapid analysis of tissue samples. In accordance with these embodiments, a tissue sample can be fresh, frozen, a homogenate or fixed archival. In addition, this simple and rapid analysis may be used to diagnosis recessive genetic disorders or a dominantly inherited genetic disorders. Examples of these disorders include but are not limited to blood diseases, muscular dystrophy and cancers such as prostate and breast cancer.

In certain embodiments, inherited mutations responsible for particular genetic disorders can include changes in a DNA molecule such as mutations, substitutions, insertions or deletions in a target molecule. In accordance with these embodiments, these changes can lead to protein truncation or post translational modifications in a target protein. In certain particular embodiments, genetic diseases such as inherited breast or colon cancer can be a consequence of mutations which alter the DNA sequence generating a termination codon which can lead to truncation of the resultant protein. Previous methods for identifying subjects with inherited disease involved expensive and cumbersome methods, for example, either complete gene sequences or PCR-based protein truncation tests which require patient blood samples and complicated and expensive technologies. One issue with these approaches is that they require a high index of suspicion that the disease exists, therefore few patients are actually identified since the genetic susceptibility is often clinically silent.

In other embodiments, a rapid, simple and inexpensive method can be used to identify tissue samples which have a specific genetic mutation, substitution, insertion or deletion leading to protein truncation as a predictor of disease. This approach facilitates a simple and rapid identification of disease mutations in a subject not previously diagnosed with a specific genetic disorder (see Table 4) nor previously suspected of having the disorder. It is contemplated herein that other screening methods using techniques, such as IHC, can be used in combination with other methods to confirm the diagnosis and assess a therapeutic treatment. Examples of these other combination technologies may include, but are not limited to, DNA sequencing, microarray technologies, tissue biopsies, ultrasound technologies, evaluation of medical history (e.g. sex, age, family history, disease history, previous health issues, current and past medications), IHC for other proteins or markers, and/or histopathologic staging. These additional methods may be used in combination with methods disclosed herein for example, to confirm the diagnosis or predict the risk of a disorder in an asymptomatic subject.

Methods herein disclose a novel approach for identifying truncated proteins by screening for absence of a portion of the protein using carboxy-terminal directed antibodies and/or amino terminal directed antibodies to a target protein. In certain embodiments, methods of the present invention employ immunohistochemical methods currently employed in many point of care laboratories. In addition, methods herein facilitate testing of all subjects whereas it was previously required that a high index of suspicion be present before pursuing further methodologies. Because diseases identified by these methods have specific molecular causes, it is likely that targeted therapies can be identified for these individuals, providing further incentive for identifying patients with truncated mutations. In certain embodiments, genetic screening can be performed on subjects without disease, for example, subjects with a strong familial history and/or those that would like to know if they may develop the disease. Exemplary methods of the present invention involve a simple and rapid screening process applicable to anyone with or suspected of developing a disorder (e.g. sporadic or genetic cancer). In addition, the methods of the present invention may identify those patients who have a genetic causation triggering family screening and carrier identification.

In some embodiments of the present invention, methods disclosed herein may be used to identify carriers of specific genetic diseases facilitating targeted therapies, family screening, and preventative strategies for identified for family members. One advantage herein is providing a simple tissue-based test to identify patients with genetic mutations. Studies have shown that over 200,000 cases of breast cancer and 106,000 cases of colon cancer occur in the U.S. each year. These are just two examples of the disorders that might benefit from the disclosed screening methods.

### Post Translational Modifications of Protein

Certain embodiments herein are directed to early detection of genetic disorders whose onset is directly linked to an alteration in one or more genes leading to an alteration in the translated protein of the gene(s). For example, alterations can include, but are not limited to, a mutation, substitution, deletion or insertion. These alterations can lead to a post translational modification (eg. phosphorylation, methylation, maturation of the protein via cleavage) of a protein or alternatively, a truncation of a protein. In addition, methods disclosed herein could be useful for example to assess the need for drug treatment, radiation therapy, physical therapy, diagnostic evaluations, preventive regimens, radiologic testing, blood and tissue testing, dialysis, surgical interventions, cardiovascular interventions, organ transplantation, blood transfusion or combination thereof

In some embodiments of the present invention, modulation of a particular post translational modification(s) of target protein(s) are investigated. In accordance with these embodiments, modulation of post translational modification is correlated with an increase or decrease in the effectiveness of potential therapies for a particular disorder. In other embodiments, modulation of post translational modification is correlated with an increase or decrease in the risk of developing or having a particular disorder. In one particular example, detection of truncation of a particular protein in a sample of a subject that correlates with a disease can be correlated with the need for a particular therapy.

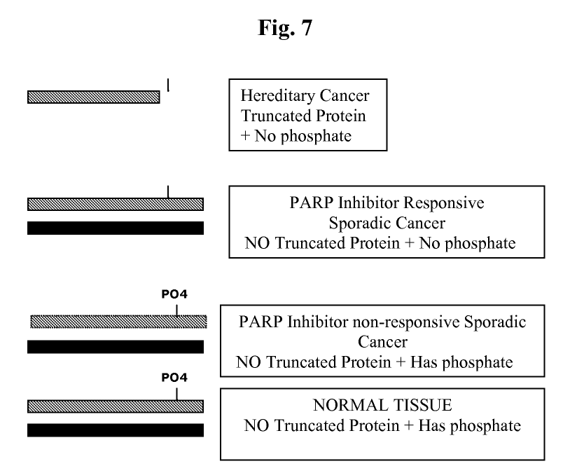

In one exemplary study, 20-40% of sporadic (non-inherited) breast cancers were found to be “BRCA2-like,” identified by a tissue test for the BRCA2 signaling defect. This exemplary test as disclosed herein can be used to quantitate phosphorylation of a particular amino acid in a target gene, for example serine 3291 of BRCA2 using a serine 3291 phosphospecific antibody. In accordance with this example, the level of post translational modification may correlate with the response of a subject to a therapeutic treatment. This therapeutic treatment example involved subjects having either a sporadic ovary or prostate cancer that can be classified as “BRCA2-like” cancers. Thus, these cancers likely respond to BRCA2 targeted therapies for example, (poly (ADP-ribose) polymerase (PARP) inhibitors. In certain particular embodiments, identifying these responders by rapidly identifying a change in a target protein might lead to an expanded number of potential responders and increased survivability of these responders. Some embodiments of the present invention may include predicting a response in a subject having or at risk of developing cancer to an anti-cancer agent, such as a PARP inhibitor (e.g. see FIG. 7). In accordance with these embodiments, one example for predicting a responder would be to use a phosphospecific antibody directed to bind a target protein of a tissue sample from the subject. In one particular example, a serine 3291 antibody directed to bind phosphorylated serine 3291 of BRCA2, would be introduced to a tissue sample of a subject. Here, reduced or absent serine 3291 antibody binding to the tissue sample of the subject can indicate that the subject may respond to agents used to treat BRCA2 cancers, for example, PARP inhibitors.

It is contemplated herein that any of the methods disclosed in the present invention used to diagnose a subject having or at risk of developing a disorder, or capable of responding to an agent to treat a particular disorder may or may not have a history linking the subject to the disorder.

Alternatively, phosphospecific antibodies contemplated herein may be used as a therapeutic and/or used to direct a therapeutic for treatment of a subject having or at risk of developing a BRCA2 cancer or a BRCA2-like cancer. It is contemplated herein that a treatment for a subject having or at risk of developing a BRCA2 cancer or a BRCA2-like cancer can include, but is not limited to, using an antibody disclosed herein to target a tumor, precancerous or cancerous population of cells in order to characterize the cells and/or deliver one or more therapeutic agents. In one particular embodiment, an antibody that specifically binds to unphosphorylated serine 3291 can be used in accordance with these embodiments.

### PARP Inhibitors

In some exemplary methods, evaluation and use of pharmaceutical agents are contemplated herein. In accordance with these embodiments, one example includes the contemplated evaluation and use of several PARP inhibitors to treat a disorder (e.g. “BRCA2-like” cancers). PARP inhibitors contemplated of use in methods of the present invention include, but are not limited to, those listed in Table 3.

### Therapeutics

In other exemplary embodiments, prediction of response to therapeutic treatments under consideration to treat a disorder may be performed using an antibody directed to identifying a change in a post-translational modification levels of a target protein associated with the disorder. In one particular embodiment, treatments of sporadic ovary or prostate cancer might include using PARP or PARP-like treatment. In one example, PARP is a nuclear protein that has an important role in DNA repair processes, notably homologous recombination. Based on specific molecular knowledge of BRCA2 function during DNA repair, an antibody-based tissue test may be used to predict which breast cancer patients will respond to PARP inhibitors. The test identifies both the uncommon hereditary BRCA2 breast cancer patients and a 10 fold more common subgroup of sporadic breast cancers patients with defective BRCA2 signaling. This test can identify breast cancer patients for PARP inhibitor therapy and can be developed as a diagnostic test to identify patients with a molecular defect targeted by PARP inhibitors.

### S3291 Antibody Development

In previous studies, analysis of cancer-associated BRCA2 mutations identified a near C-terminal region of the protein used for tumor suppression. This region precisely corresponds to a cdk2-regulated BRCA2 phosphorylation site which appears to mediate binding to Rad51 protein and mediate homologous recombination type DNA repair. An antibody was developed which specifically recognizes BRCA2 serine 3291 phosphorylation. Data revealed that this antibody binds to no protein by western blotting or immunohistochemistry in samples from hereditary cancer samples. In addition, ionizing radiation was demonstrated to markedly decrease S3291 phosphorylation of BRCA2 rescued cell lines. This exemplary antibody is specific for BRCA2 phosphorylated on serine 3291 for both western blotting and immunohistochemistry (IHC). Nearly all of the IHC signal with this antibody on normal breast tissue is blocked by phosphorylated peptide from this region but not by unphosphorylated peptide from this region. In one exemplary embodiment, formalin-fixed, paraffin embedded tissues from breast cancer patients were exposed to the antibody and demonstrated that this phosphorylation is essentially non-existent in BRCA2 hereditary cancers and about 40-50% of estrogen receptor positive (ER+) sporadic breast cancers tested to date. These sporadic cancers would likely benefit from BRCA2 targeted therapy.

In one embodiment, an antibody may be generated and used in any of the methods disclosed herein. It is contemplated herein that an antibody can be developed by methods disclosed herein that targets the presence of a specific post-translational modification or targets the reduction or absence of a specific post-translational modification of a target protein. One exemplary method of the present invention includes the generation of a polyclonal phosphoserine specific antibody of BRCA2, namely serine 3291 (S3291) antibodies. Rabbits (e.g. New Zealand white rabbits) were immunized with a phosphorylated peptide TFVSPAAKAGG (SEQ ID NO:1) conjugated to an immunogen (e.g. Keyhole Limpet Hemocyanin (KLH)). The antisera from the rabbit was collected and the antibodies were affinity purified by selective elution with phosphorylated and unphosphorylated peptide bound to sepharose. The antibodies were further purified by methods known in the art. These antibodies or other antibodies, such as a monoclonal antibody, may be used alone or in combination with any of the methods disclosed herein. For tissue sample analysis, for example, analyzing for the presence or absence of a phosphorylated amino acid can be performed. In one particular embodiment, it is contemplated that as few as 3 consecutive amino acids of SEQ ID NO:1 may be used to generate an antibody specific to bind phosphorylated serine 3291 of BRCA2.

In other embodiments disclosed herein, one or more antibodies can be used to assess changes in proteins associated with the diagnosis of or predisposition of an inherited or metabolic disease. In one particular example, the disease can be muscular dystrophy. In accordance with these embodiments, one or more antibodies or antibody fragments can be used to assess a mutation in a protein associated with muscular dystrophy. In certain embodiments, one or more tissue samples may be obtained from a subject and a target protein of the sample(s) can be analyzed for binding by one or more antibodies including but not limited to the carboxy-terminal antibodies. In a more particular embodiment, one protein associated with muscular dystrophy can be dystrophin. In this particular example, a mutation associated with dystrophin can be a mutation associated with truncation of the protein. Thus, in certain embodiments, antibodies or similar detection molecules can be used to assess the presence, absence or level of truncated dystrophin. In a more particular embodiment, an increase in truncation of dystrophin in the carboxy terminus can be associated with an increase likelihood of muscular dystrophy.

### Combination Methodologies

In some embodiments herein, one or more phosphospecific antibodies can be used to screen a tissue sample from a subject for the level of phosphorylated amino acids of one or more target proteins known to associate with a genetic disorder, to evaluate the potential response of a subject to a predetermined therapeutic treatment for the disorder. In one particular embodiment, a phosphospecific antibody can be used to screen a tissue sample of a subject for response to a therapy, for example PARP inhibitors including, but not limited to, 3-amino-benzamide, 8-hydroxy-2-methylquinazolin-4-(3H)-one (NU1025), AG14361. In another embodiment herein, one or more phosphospecific antibodies can be used to screen a tissue sample from a subject for the level of target phosphorylated amino acids of one or more target proteins, in order to evaluate the response of a subject to a predetermined therapeutic treatment in addition to screening a tissue sample with a carboxy-terminal antibody to identify a modification in a protein known to cause truncation of the same or different tissue-associated target protein. In one example, using a C-terminal antibody, truncated BRCA2 proteins may be identified that correlate with a risk for hereditary cancers in combination with using a phosphospecific antibody to detect phosphorylated amino acids in BRCA2. In this particular example, hereditary cancers would be negative for both phosphospecific and C-terminal antibody, but non-inherited PARP-inhibitor responsive sporadic cancers would be negative only for the phosphospecific antibody (since there is no truncating mutation deleting the C-terminus) See for example, the schematic in FIG. 7. Therefore, subjects having a sporadic cancer may be candidates for BRCA2 therapies. In one example, these therapies can include PARP.

In one embodiment the level of binding of a phosphospecific antibody to a tissue sample target protein can be assessed using a rapid screening technique. Certain particular examples of these techniques include, but are not limited to, immunohistochemistry, western blot analysis, Elisa, immunoprecipitation, radioimmunoassay, mass spectroscopy, gas-chromatography-mass spectroscopy, two-dimensional electrophoresis and staining with organic dyes, metal chelates, fluorescent dyes, complexing with silver, or pre-labeling with fluorophores, as well as any future technology capable of ascertaining the level of phosphospecific antibody binding to a sample. In accordance with these embodiments, the level of phosphospecific antibody may be used to distinguish genetic variations of a disorder such as a blood disorder or cancer. A better understanding of the genetic variation of a disorder can lead to a more accurate diagnosis and prognosis as well as a more tailored therapeutic treatment for a subject having or suspected of developing a disorder.

In other embodiments, the techniques disclosed herein may be used as an initial screening process on a subject having or at risk of developing a disorder. Upon completion of the screening of a subject for phosphospecific antibody binding to a target protein and/or detection of truncation of a target protein, the levels of binding of the antibodies to a tissue sample may be used to evaluate whether further testing is necessary, immediate intervention is required or future evaluations and monitoring are needed by a healthcare professional to evaluate the subject.

An “antibody” as used herein refers to a full-length (i.e., naturally occurring or formed by normal immunoglobulin gene fragment recombinatorial processes) immunoglobulin molecule (eg., an IgG antibody) or an immunologically active (i.e., specifically binding) portion of an immunoglobulin molecule, for example, an antibody fragment. The term “antibody” also includes “humanized” antibodies and even fully human antibodies that can be produced by phage display, gene and chromosome transfection methods, as well as by other means. This term also includes monoclonal antibodies, polyclonal antibodies, multivalent antibodies, multispecific antibodies (e.g., bispecific antibodies).

Naturally occurring (wild type) antibody molecules are Y-shaped molecules consisting of four polypeptide chains, two identical heavy chains and two identical light chains, which are covalently linked together by disulfide bonds. Both types of polypeptide chains have constant regions, which do not vary or vary minimally among antibodies of the same class (i.e., IgA, IgM, etc.), and variable regions. The variable regions are unique to a particular antibody and comprise a recognition element for an epitope. The carboxy-terminal regions of both heavy and light chains are conserved in sequence and are called the constant regions (also known as C-domains). The amino-terminal regions (also known as V-domains) are variable in sequence and are responsible for antibody specificity. The antibody specifically recognizes and binds to an antigen mainly through six short complementarity-determining regions (CDRs) located in their V-domains.

Each light chain of an antibody is associated with one heavy chain, and the two chains are linked by a disulfide bridge formed between cysteine residues in the carboxy-terminal region of each chain, which is distal from the amino terminal region of each chain that constitutes its portion of the antigen binding domain. Antibody molecules are further stabilized by disulfide bridges between the two heavy chains in an area known as the hinge region, at locations nearer the carboxy terminus of the heavy chains than the locations where the disulfide bridges between the heavy and light chains are made. The hinge region also provides flexibility for the antigen-binding portions of an antibody.

The antigen-binding specificity of an antibody can be determined by its variable regions located in the amino terminal regions of the light and heavy chains. The variable regions of a light chain and associated heavy chain form an “antigen binding domain” that recognizes a specific epitope; an antibody thus has two antigen binding domains. The antigen binding domains in a wild type antibody are directed to the same epitope of an immunogenic protein, and a single wild type antibody is thus capable of binding two molecules of the immunogenic protein at the same time. Thus, a wild type antibody is monospecific (i.e., directed to a unique antigen) and divalent (i.e., capable of binding two molecules of antigen).

“Polyclonal antibodies” are generated in an immunogenic response to a protein having many epitopes. A composition (e.g., serum) of polyclonal antibodies thus includes a variety of different antibodies directed to the same and to different epitopes within the protein. Methods for producing polyclonal antibodies are known in the art.

“Antipeptide antibodies” (also known as “monospecific antibodies”) are generated in a humoral response to a short (typically, 5 to 20 amino acids) immunogenic polypeptide that corresponds to a few (preferably one) isolated epitopes of the protein from which it is derived. A plurality of antipeptide antibodies includes a variety of different antibodies directed to a specific portion of the protein, i.e., to an amino acid sequence that contains at least one, preferably only one, epitope. Methods for producing antipeptide antibodies are known in the art.

A “monoclonal antibody” is a specific antibody that recognizes a single specific epitope of an immunogenic protein. In a plurality of a monoclonal antibody, each antibody molecule is identical to the others in the plurality. In order to isolate a monoclonal antibody, a clonal cell line that expresses, displays and/or secretes a particular monoclonal antibody is first identified; this clonal cell line can be used in one method of producing the antibodies of the present invention. Methods for the preparation of clonal cell lines and of monoclonal antibodies expressed thereby are known in the art.

A “naked antibody” is an intact antibody molecule that contains no further modifications such as conjugation with a toxin, or with a chelate for binding to a radionuclide. The Fc portion of the naked antibody can provide effector functions, such as complement fixation and ADCC (antibody dependent cell cytotoxicity), which set mechanisms into action that may result in cell lysis. These methods are known in the art.

In another embodiment, the Fc portion may not be needed or in some instances desired for a therapeutic treatment of a subject. In accordance with this embodiment, other mechanisms, such as apoptosis, may be invoked.

An “antibody fragment” is a portion of an intact antibody such as F(ab′)a, F(ab)2, Fab′, Fab, Fv, sFv and the like. Regardless of structure, an antibody fragment binds with the same antigen that is recognized by the full-length antibody. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

Antibody fragments produced by limited proteolysis of wild type antibodies are called proteolytic antibody fragments. These include, but are not limited to, the following: “F(ab′)₂ fragments” are released from an antibody by limited exposure of the antibody to a proteolytic enzyme, e.g. pepsin or ficin. An F(ab′)2 fragment comprises two “arms,” each of which comprises a variable region that is directed to and specifically binds a common antigen. The two Fab′ molecules are joined by interchain disulfide bonds in the hinge regions of the heavy chains; the Fab′ molecules may be directed toward the same (bivalent) or different (bispecific) epitopes.

“Fab′-SH fragments” are typically produced from F(ab′)2 fragments, which are held together by disulfide bond(s) between the H chains in an F(ab′)2 fragment. Treatment with a mild reducing agent such as, by way of non-limiting example, beta-mercaptoethylamine, breaks the disulfide bond(s), and two Fab′ fragments are released from one F(ab′)₂ fragment. Fab′-SH fragments are monovalent and monospecific.

“Fab fragments” (i.e., an antibody fragment that contains the antigen-binding domain and comprises a light chain and part of a heavy chain bridged by a disulfide bond) are produced by papain digestion of intact antibodies. A convenient method is to use papain immobilized on a resin so that the enzyme can be easily removed and the digestion terminated. Fab fragments do not have the disulfide bond(s) between the H chains present in an F(ab′)2 fragment.

“Single-chain antibodies” are one type of antibody fragment. The term single chain antibody is often abbreviated as “scFv” or “sFv.” These antibody fragments are produced using molecular genetics and recombinant DNA technology. A single-chain antibody consists of a polypeptide chain that comprises both a VH and a VL domains which interact to form an antigen-binding site. The VH and VL domains are usually linked by a peptide of 10 to 25 amino acid residues.

The term “single-chain antibody” further includes but is not limited to a disulfide-linked Fv (dsFv) in which two single-chain antibodies (each of which may be directed to a different epitope) linked together by a disulfide bond; a bispecific sFv in which two discrete scFvs of different specificity is connected with a peptide linker; a diabody (a dimerized sFv formed when the VH domain of a first sFv assembles with the VL domain of a second sFv and the VL domain of the first sFv assembles with the VH domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes).

“Complementary determining region peptides” or “CDR peptides” are another form of an antibody fragment. A CDR peptide (also known as “minimal recognition unit”) is a peptide corresponding to a single complementarity-determining region (CDR), and can be prepared by constructing genes encoding the CDR of an antibody of interest. Such genes are prepared, for example, by using the polymerase chain reaction to synthesize the variable region from RNA of antibody-producing cells. These are methods are known in the art.

In “cysteine-modified antibodies,” a cysteine amino acid is inserted or substituted on the surface of antibody by genetic manipulation and used to conjugate the antibody to another molecule via, e.g. a disulfide bridge. Cysteine substitutions or insertions for antibodies have been described. Methods for introducing Cys residues into the constant region of the IgG antibodies for use in site-specific conjugation of antibodies have been described.

A “humanized antibody” is a recombinant protein used to reduce the amount of non-human protein in which the CDRs from an antibody from one species; eg. a rodent antibody, heavy and light variable chains of the rodent antibody are exchanged for some human heavy and light variable domains for example using protein engineering techniques. The constant domains of the antibody molecule are derived from those of a human antibody. See Gussow and Seemann, Humanization of monoclonal antibodies are known in the art.

### Production of Antibody Fragments

Some embodiments of the claimed methods and/or compositions may concern antibody fragments. Such antibody fragments may be obtained by pepsin or papain digestion of whole antibodies by conventional methods. For example, antibody fragments may be produced by enzymatic cleavage of antibodies with pepsin to provide a 5S fragment denoted F(ab′)₂. This fragment may be further cleaved using a thiol reducing agent and, optionally, a blocking group for the sulfhydryl groups resulting from cleavage of disulfide linkages, to produce 3.5S Fab′ monovalent fragments. Alternatively, an enzymatic cleavage using pepsin produces two monovalent Fab fragments and an Fc fragment. Exemplary methods for producing antibody fragments are known in the art.

Other methods of cleaving antibodies, such as separation of heavy chains to form monovalent light-heavy chain fragments, further cleavage of fragments or other enzymatic, chemical or genetic techniques also may be used, so long as the fragments bind to the antigen that is recognized by the intact antibody. For example, Fv fragments comprise an association of VH and VL chains. This association can be noncovalent. Alternatively, the variable chains may be linked by an intermolecular disulfide bond or cross-linked by chemicals such as glutaraldehyde by techniques known in the art.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. These techniques are known in the art.

In another embodiment of the present invention, the humanized antibody may include a complete antibody molecule, having full length heavy and light chains; a fragment thereof, such as a Fab, Fab′, F(ab′)2, or Fv fragment; a single chain antibody fragment, e.g. a single chain Fv, a light chain or heavy chain monomer or dimer; multivalent monospecific antigen binding proteins comprising two, three, four or more antibodies or fragments thereof bound to each other by a connecting structure; or a fragment or analogue of any of these or any other molecule with the same specificity as a phosphospecific antibody, carboxy-terminal or amino-terminal binding antibody. In one particular embodiment, the antibody may include a complete antibody molecule, having full length heavy and light chains.

### Preparation of Genes Encoding Phosphospecific or C-Terminal or N-Terminal Directed Antibodies

Any standard technique of molecular biology known in the art may be used to prepare DNA sequences coding for the antibodies according to the present invention. For example, DNA sequences may be synthesised completely or in part using oligonucleotide synthesis techniques. Site-directed mutagenesis and polymerase chain reaction (PCR) techniques may be used as appropriate. Suitable processes include the PCR strand overlap procedure and PCR mutagenesis as described in for example “PCR Technology Principles and Applications for DNA Amplification” (1989), Ed. H. A. Erlich, Stockholm Press, N.Y., London, and oligonucleotide directed mutagenesis (Kramer et al, Nucleic. Acid. Res. 12 9441 (1984)).

An “expression vector” as used herein is a DNA molecule including the genes of interest that are expressed in a host cell. Typically, gene expression is placed under the control of certain regulatory elements, including constitutive or inducible promoters, tissue-specific regulatory elements and enhancers. Such a gene is said to be “operably linked to” the regulatory elements. In further aspects, one embodiment also includes DNA sequences coding for the heavy and light chains of the antibodies of the present invention, cloning and expression vectors containing these DNA sequences, host cells transformed with these DNA sequences and processes for producing the heavy or light chains or full length antibody and antibody molecules comprising expressing these DNA sequences in a transformed host cell. In some embodiments of the present invention, expression vectors may be required to express large quantities of the phosphospecific, c-terminal or N-terminal antibodies within a host producing cell.

DNA coding for human immunoglobulin sequences may be obtained by any means known in the art. The skilled artisan is aware that multiple codon sequences may encode the same amino acid and that in various embodiments, the disclosed nucleic acid sequences may be substituted with an alternative sequence that encodes the same sequence of amino acids. The skilled artisan is also aware that, depending on the species of origin for a cell line used to express a protein from a nucleic acid sequence, the codon usage may be optimized to enhance expression in the selected species. Such species preferred codon frequencies are well known in the art.

In one embodiment, the antibody disclosed herein may be a complete antibody, or as explained above, a fragment thereof, a monomer or dimer or a multivalent monospecific antigen binding protein. Thus, further to one aspect of the present invention, a multivalent monospecific antigen binding protein may be provided comprising two, three, four or more antibodies fragments thereof bound to each other by a connecting structure, which protein is not a natural immunoglobulin, each of said antibodies or fragments having a specificity for the epitope recognized by a phosphospecific antibody, said antigen binding protein being optionally conjugated with an effector or reporter molecule.

In accordance with these embodiments, each antibody or fragment may be a humanized antibody or a fragment thereof, as defined above, and a multivalent monospecific antigen binding protein may be a humanized multivalent monospecific antigen binding protein. Non-humanized, e.g., murine, multivalent monospecific antigen binding proteins, however, may be contemplated and an embodiment may extend to these where applicable.

### General Methods for the Production of Recombinant Fusion Proteins Containing Antibody Fragments

Nucleic acid sequences encoding antibody fragments that recognize specific epitopes can be obtained by techniques that are well known in the art. For example, hybridomas secreting antibodies of a desired specificity can be used to obtain antibody-encoding DNA that can be prepared using known techniques, for example, by PCR or by traditional cDNA cloning techniques. Alternatively, Fab′ expression libraries or antibody phage display libraries can be constructed to screen for antibody fragments having a desired specificity.

Proteins or peptides may be synthesized, in whole or in part, in solution or on a solid support in accordance with conventional techniques. Various automatic synthesizers are commercially available and can be used in accordance with known protocols. See, for example, Stewart and Young, (1984, Solid Phase Peptide Synthesis, 2d. ed., Pierce Chemical Co.); Tam et al., (1983, J. Am. Chem. Soc., 105:6442); Merrifield, (1986, Science, 232: 341-347); and Barany and Merrifield (1979, The Peptides, Gross and Meienhofer, eds., Academic Press, New York, pp. 1-284). Short peptide sequences, usually from about 6 up to about 35 to 50 amino acids, can be readily synthesized by such methods. Alternatively, recombinant DNA technology may be employed wherein a nucleotide sequence which encodes a peptide of interest is inserted into an expression vector, transformed or transfected into an appropriate host cell, and cultivated under conditions suitable for expression.

Methods for producing recombinant proteins in a desired host cell are well known in the art. To facilitate purification, the stably tethered structures may contain suitable peptide tags, such as the FLAG sequence or the poly-HIS sequence, to facilitate their purification with a relevant affinity column.

In one embodiment, the Fv fragments may include VH and VL chains connected by a peptide linker. These single-chain antigen binding proteins (sFv) are prepared by constructing a structural gene comprising DNA sequences encoding the VH and VL domains, connected by an oligonucleotides linker sequence. The structural gene is inserted into an expression vector that is subsequently introduced into a host cell, such as E. coli. The recombinant host cells synthesize a single polypeptide chain with a linker peptide bridging the two V domains. Methods for producing sFvs are well-known in the art.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells.

Suitable host cells or cell lines for the expression of the constituent subunits of the stably tethered structures of are known to one skilled in the art. The use of a human host cell would enable any expressed molecules to be modified with human glycosylation patterns. However, there is no indication that a human host cell is essential or preferred for the disclosed methods

In one embodiment, the antibody disclosed herein may be a complete antibody, or as explained above, a fragment thereof, a monomer or dimer or a multivalent monospecific antigen binding protein.

Suitable host cells or cell lines for the expression of the antibodies disclosed in the present invention are known in the art.

An example of a therapeutic agent for use in a therapeutic treatment is a molecule or atom which is administered to a subject in need of such a therapy. Examples of therapeutic agents include antibodies, antibody fragments, drugs, toxins, enzymes, nucleases, hormones, immunomodulators, oligonucleotides, interference RNA, chelators, boron compounds, photoactive agents or dyes and radioisotopes.

Useful diagnostic/detection agents for use in combination technologies disclosed herein include, but are not limited to, radioisotopes, dyes (such as with the biotin-streptavidin complex), radiopaque materials (e.g., iodine, barium, gallium, and thallium compounds and the like), contrast agents, fluorescent compounds or molecules and enhancing agents (e.g., paramagnetic ions) for magnetic resonance imaging (MRI). U.S. Pat. No. 6,331,175 describes MRI technique and the preparation of antibodies conjugated to a MRI enhancing agent and is incorporated in its entirety by reference. Preferably, the diagnostic/detection agents are selected from the group consisting of radioisotopes for nuclear imaging, intraoperative and endoscopic detection; enhancing agents for use in magnetic resonance imaging or in ultrasonography; radiopaque and contrast agents for X-rays and computed tomography; and fluorescent compounds for fluoroscopy, including endoscopic fluoroscopy.

Chemotherapeutic agents, for the purpose of this disclosure that may be used alone or in combination with other disclosed therapies, include all known chemotherapeutic agents. Known chemotherapeutic agents include but are not limited to the taxanes, nitrogen mustards, ethylenimine derivatives, alkyl sulfonates, nitrosoureas, triazenes, folic acid analogs, pyrimidine analogs, purine analogs, antisense oligonucleotides, antagonists or inhibitors of transcription factors, interference RNAs, alkaloids, antibiotics, enzymes, platinum coordination complexes, COX-2 inhibitors, apoptotic agents, substituted urea, methyl hydrazine derivatives, adrenocortical suppressants, or antagonists. In a more particular embodiment, the chemotherapeutic agents may include steroids, progestins, estrogens, antiestrogens, or androgens. In another particular embodiment, the chemotherapy agents may include actinomycin, azaribine, anastrozole, azacytidine, bleomycin, bryostatin-1, busulfan, carmustine, Celebrex, chlorambucil, cisplatin, irinotecan (CPT-11), carboplatin, cladribine, cyclophosphamide, cytarabine, dacarbazine, docetaxel, dacarbazine, dactinomycin, daunorubicin, dexamethasone, diethylstilbestrol, doxorubicin, ethinyl estradiol, estramustine, etoposide, floxuridine, fludarabine, flutamide, fluorouracil, fluoxymesterone, gemcitabine, hydroxyprogesterone caproate, hydroxyurea, idarubicin, ifosfamide, L-asparaginase, leucovorin, lomustine, mechlorethamine, medroprogesterone acetate, megestrol acetate, melphalan, mercaptopurine, methotrexate, mitoxantrone, rnithramycin, mitomycin, mitotane, oxaliplatin, phenyl butyrate, prednisone, procarbazine, paclitaxel, pentostatin, semustine streptozocin, SN-38, tamoxifen, taxanes, taxol, testosterone propionate, thalidomide, thioguanine, thiotepa, teniposide, topotecan, uracil mustard, vinblastine, vinorelbine or vincristine.

Some suitable chemotherapeutic agents are described in Remington's Pharmaceutical Sciences 19th Ed. (Mack Publishing Co. 1995). Other suitable chemotherapeutic agents, such as experimental drugs, are known to those of skill in the art.

In one embodiment of the present invention, a toxin may include but is not limited to ricin, abrin, ribonuclease, DNase I, Staphylococcal enterotoxin-A, pokeweed antiviral protein, gelonin, diphtherin toxin, Pseudomonas exotoxin, or Pseudomonas endotoxin.

In one embodiment of the present invention, enzymes are also useful therapeutic agents and may be selected from the group including but not limited to malate dehydrogenase, Staphylococcal nuclease, delta-V-steroid isomerase, yeast alcohol dehydrogenase, a-glycerophosphate dehydrogenase, triose phosphate isomerase, horseradish peroxidase, alkaline phosphatase, asparaginase, glucose oxidase, p-galactosidase, ribonuclease, urease, catalase, glucose-6-phosphate dehydrogenase, glucoamylase and acetylcholinesterase.

In one embodiment of the present invention, immunomodulators are also useful therapeutic agents that include cytokines, stem cell growth factors, lymphotoxins, such as tumor necrosis factor (TNF), and hematopoietic factors, such as interleukins (e.g., interleukin-1 (IL-1), IL-2, IL-3, IL-6, IL-10, IL-12, IL-18 and IL-21), colony stimulating factors (e.g., granulocyte-colony stimulating factor (G-CSF) and granulocyte macrophage-colony stimulating factor (GM-CSF)), interferons (e.g., interferons-α, -β and -γ), the stem cell growth factor designated “S1 factor”, and erythropoietin and thrombopoietin. Examples of suitable immunomodulator moieties include IL-2, IL-6, IL-10, IL-12, IL-18, IL-21, interferon-γ, TNF-α, and the like. Alternatively, subjects can receive invention compositions and a separately administered cytokine, which can be administered before, concurrently or after administration of compositions disclosed herein.

A “diagnostic/detection agent” is a molecule or atom which is administered linked to or conjugated to an antibody moiety, i.e., antibody or antibody fragment, or subfragment, and is useful in diagnosing or detecting a disease by locating the cells containing the antigen. Useful diagnostic/detection agents include, but are not limited to, radioisotopes, dyes (such as with the biotin-streptavidin complex), contrast agents, fluorescent compounds or molecules and enhancing agents (e.g. paramagnetic ions) for magnetic resonance imaging (MRI), and particles or liposomes as examples of agents used for ultrasound imaging. In one embodiment of the present invention, it is contemplated that antibody compositions disclosed herein may be conjugated to a diagnostic or detection agent and administered to a subject in need of an evaluation or targeted treatment.

It is contemplated herein that any of the disclosed antibodies may be used alone or in combination to detect the presence of the target protein modification associated with a tissue. In addition, it is contemplated that the antibodies disclosed herein may be used to direct a therapeutic agent to a specific tissue alone or in combination with other antibodies directed to deliver the same or a different therapeutic agent.

### Nucleic Acids

As described herein, an aspect of the present disclosure concerns isolated nucleic acids and methods of use of isolated nucleic acids. The term “nucleic acid” is intended to include DNA and RNA and can be either be double-stranded or single-stranded. In a preferred embodiment, the nucleic acid is a cDNA comprising a nucleotide sequence such as found in GenBank. In certain embodiments, the nucleic acid sequences disclosed herein have utility as hybridization probes or amplification primers. These nucleic acids may be used, for example, in diagnostic evaluation of tissue samples. In certain embodiments, these probes and primers consist of oligonucleotide fragments. Such fragments should be of sufficient length to provide specific hybridization to a RNA or DNA tissue sample. The sequences typically will be 10-20 nucleotides, but may be longer. Longer sequences greater than 50 even up to full length, are preferred for certain embodiments.

Accordingly, the nucleotide sequences may be used for their ability to selectively form duplex molecules with complementary stretches of genes or RNAs or to provide primers for amplification of DNA or RNA from tissues. Those that are skilled in the art know the stringency needed for effective hybridization of the complementary component.

Many of the genes associated with a genetic disorder (Appendix B) have been cloned. Embodiments of this invention include use of these known as well as those unknown genes associated with predisposition to a genetic disorder for predicting onset or risk of the disorder using a rapid tissue analysis test disclosed herein Any of these genes may be used to generate unique antibodies for detecting mutations or mutational changes in an expressed protein.

The gene or gene fragment encoding a polypeptide (e.g. BRCA2) may be inserted into an expression vector by standard subcloning techniques. An E. coli expression vector may be used which produces the recombinant polypeptide as a fusion protein, allowing rapid affinity purification of the protein. Examples of such fusion protein expression systems are the FLAG system (IBI, New Haven, Conn.), and the 6xHis system (Qiagen, Chatsworth, Calif.).

Inducible non-fusion expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). While target gene expression relies on host RNA polymerase transcription from the hybrid trp-lac fusion promoter in pTrc, expression of target genes inserted into pET ld relies on transcription from the T7 gn10-lac 0 fusion promoter mediated by coexpressed viral RNA polymerase (T7 gnl). This viral polymerase is supplied by host strains BL21 (DE3) or HMS174(DE3) from a resident lambda prophage harboring a T7 gnl under the transcriptional control of the lacUV 5 promoter.

Expression of a genetic disorder associated protein in mammalian cells may be accomplished using a mammalian expression vector. Examples of mammalian expression vectors include pCDM8 (Seed, B., (1987) Nature 329:840) and pMT2PC (Kaufman et al. (1987), EMBO J. 6:187-195).

Plasmid vectors introduced into mammalian cells are integrated into host cell DNA at only a low frequency. In order to identify these integrants, a gene that contains a selectable marker (i.e., resistance to antibiotics) is generally introduced into the host cells along with the gene of interest. Preferred selectable markers include those that confer resistance to certain drugs, such as G418 and hygromycin. Selectable markers can be introduced on a separate plasmid from the nucleic acid of interest or, preferably, are introduced on the same plasmid. Host cells transformed with one or more recombinant expression vectors containing a nucleic acid and a selectable marker may be identified by locating the marker. For example, if the selectable marker encoded a gene conferring neomycin resistance (such as pRc/CMV), transformant cells can be selected with G418. Cells that have incorporated the selectable marker gene will survive, while the other cells die.

For applications in which the nucleic acid segments are incorporated into vectors, such as plasmids, cosmids or viruses, these segments may be combined with other DNA sequences, such as promoters, polyadenylation signals, restriction enzyme sites, multiple cloning sites, other coding segments, and the like, such that their overall length may vary considerably.

The recombinant expression vectors can be designed for expression of proteins in prokaryotic or eukaryotic cells. For example, proteins can be expressed in bacterial cells such as E. coli, insect cells (using baculovirus), yeast cells or mammalian cells.

One embodiment includes isolated nucleic acids encoding proteins having biological activity of a genetic disorder-associated protein. The term “isolated” refers to a nucleic acid substantially free of cellular material or culture medium when produced by recombinant DNA techniques, or chemical precursors or other chemicals when chemically synthesized. An “isolated” nucleic acid is also free of sequences that naturally flank the nucleic acid (i.e., sequences located at the 5′ and 3′ ends of the nucleic acid) in the organism from which the nucleic acid is derived.

It will be appreciated that isolated nucleic acids includes nucleic acids having substantial sequence homology with the nucleotide sequence of a genetic disorder-associated protein found in GenBank as disclosed in methods found herein or encoding proteins having substantial homology to the corresponding amino acid sequence (e.g. 60%, 70% or 80% homology). Proteins comprising an amino acid sequence that is 50 %, 60%, 70%, 80% or 90% homologous with the amino acid may provide proteins having a genetic disorder-associated protein activity.

A nucleic acid of the embodiments, for instance an oligonucleotide, can also be chemically synthesized using standard techniques. Various methods of chemically synthesizing polydeoxynucleotides are known, including solid-phase synthesis which, like peptide synthesis, has been fully automated in commercially available DNA synthesizers (See i.e., Itakura et al. U.S. Pat. No. 4,598,049; Caruthers et al. U.S. Pat. No. 4,458,066; and Itakura U.S. Pat. Nos. 4,401,796 and 4,373,071).

### Protein Purification

Various methods for quantifying the degree of purification of the protein or peptide will be known to those of skill in the art in light of the present disclosure. These include, for example, determining the specific activity of an active fraction, or analysis by SDS/PAGE to identify the number of polypeptides in a given fraction. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will be dependent upon the particular assay technique chosen to follow the purification and whether or not the expressed protein or peptide exhibits a detectable activity.

Methods for purifying various forms of proteins are known. (i.e., Protein Purification, ed. Scopes, Springer-Verlag, New York, N.Y., 1987; Methods in Molecular Biology: Protein Purification Protocols, Vol. 59, ed. Doonan, Humana Press, Totowa, N.J., 1996). The methods disclosed in the cited references are exemplary only and any variation known in the art may be used. Where a protein is to be purified, various techniques may be combined, including but not limited to cell fractionation, column chromatography (e.g., size exclusion, ion exchange, reverse phase, affinity, etc.), Fast Performance Liquid Chromatography (FPLC), High Performance Liquid Chromatography (HPLC), gel electrophoresis, precipitation with salts, pH, organic solvents or antibodies, ultrafiltration and/or ultracentrifugation.

There is no general requirement that the protein or peptide always be provided in the most purified state. Indeed, it is contemplated that less substantially purified products will have utility in certain embodiments. Partial purification may be accomplished by using fewer purification steps in combination, or by utilizing different forms of the same general purification scheme. Methods exhibiting a lower degree of relative purification may have advantages in total recovery of protein product, or in maintaining the activity of an expressed protein.

### Pharmaceutical Compositions

Embodiments herein provide for administration of compositions to subjects in a biologically compatible form suitable for pharmaceutical administration in vivo. By “biologically compatible form suitable for administration in vivo” is meant a form of the active agent (i.e. pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

In one embodiment, the compound (e.g. a pharmaceutical chemical, protein, peptide, antibody etc. of the embodiments) may be administered in a convenient manner such as subcutaneous, intravenous, by oral administration, inhalation, transdermal application, intravaginal application, topical application, intranasal or rectal administration. Depending on the route of administration, the active compound may be coated in a material to protect the compound from the degradation by enzymes, acids and other natural conditions that may inactivate the compound.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

The active therapeutic agents may be formulated within a mixture to comprise about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition.

In another embodiment, nasal solutions or sprays, aerosols or inhalants may be used to deliver the compound of interest. Additional formulations that are suitable for other modes of administration include suppositories and pessaries. A rectal pessary or suppository may also be used. In general, for suppositories, traditional binders and carriers may include, for example, polyalkylene glycols or triglycerides; such suppositories may be formed from mixtures containing the active ingredient in the range of 0.5% to 10%, preferably 1% 2%.

It will be apparent that, for any particular subject, specific dosage regimens may be adjusted over time according to the individual need. The preferred doses for administration can be anywhere in a range between about 0.01 mg and about 100 mg per ml of biologic fluid of treated patient. In one particular embodiment, the range can be between 1 and 100 mg/kg which can be administered daily, every other day, biweekly, weekly, monthly etc. In another particular embodiment, the range can be between 10 and 75 mg/kg introduced weekly to a subject.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent.

In certain embodiments of the methods of the present invention, the subject may be a mammal such as a human or a non-human such as a wild animal, bird, reptile, a domesticated animal or farm animal.

### Kits

In still further embodiments, the present invention concerns kits for use with the methods described above. Small molecules, proteins, antibodies or peptides may be employed for use in any of the disclosed methods. In addition, other agents such as anti-bacterial agents, immunosuppressive agents, anti-inflammatory agents may be provided in the kit. The kits will thus can include, in suitable container means, a protein or a peptide or analog agent, and optionally one or more additional agents.

The kits may further include a suitably aliquoted composition of the antibody, encoded protein or polypeptide antigen, whether labeled or unlabeled, as may be used to prepare a standard curve for a detection assay. The kits may further include positive and negative controls.

The container means of the kits will generally include at least one vial, test tube, flask, bottle, syringe or other container means, into which the antibody or antigen may be placed, and preferably, suitably aliquoted. Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

In one exemplary technique, a method was developed to identify tissue samples that have specific gene mutations leading to protein truncation. In one example, using methods disclosed herein, protein truncating mutations in the BRCA2 gene were identified. In one particular example, immunohistochemistry of a tissue sample using a C-terminal antibody to BRCA2 was performed. Here it was demonstrated that cancers from patients with BRCA2 mutations show no immune staining for the protein C-terminus (due to the protein truncation) while normal tissues from these patient and control cancers and normal tissues without BRCA2 mutations all show positive immunostaining. Because recessive mutations leading to truncations of the BRCA2 protein lead to a recessive genetic disease called Fanconi's anemia, this method could also be used to diagnose other recessive genetic disorders. In addition, this method could be used be employed to demonstrate protein truncation of any highly expressed protein for which a C-terminal antibody is available or capable of generating.

In one exemplary method, a study to determine whether immunohistochemistry (IHC) using N-terminal and C-terminal antibodies could be used to distinguish BRCA2 hereditary cancers and sporadic breast cancers was performed. In this study, the group included 3 patients with BRCA2 hereditary breast cancer (specific mutations were 6174delT, 6174delT, and R2520X), 3 patients with BRCA1 hereditary breast cancer, and 20 patients with sporadic breast cancer. The study was conducted in a blinded manner so the technician performing the staining and the pathologist reading the slides were unaware which samples were sporadic and which were inherited cancers. Tissue sections were cut from paraffin blocks and heat inactivated in a decloaking chamber for 5 minutes in sodium citrate buffer pH 6.0 to allow antigen retrieval. The antibodies used for this example were rabbit polyclonal antibodies to N-terminal amino acids (R and D systems) and C-terminal amino acids 3245-3418 of BRCA2 (Oncogene Research). The sample results are summarized in Table 1:

TABLE 1
Result of IHC of breast cancers with C-terminal BRCA2 Antibody.

| | Negative staining | Positive staining |
|---|---|---|
| Cancer Type | (Positive Truncation Test) | (Negative Truncation Test) |
| BRCA 2 | 3 | 0 |
| Hereditary | | |
| BRCA 1 | 0 | 3 |
| Hereditary | | |
| SPORADIC | 0 | 20 |

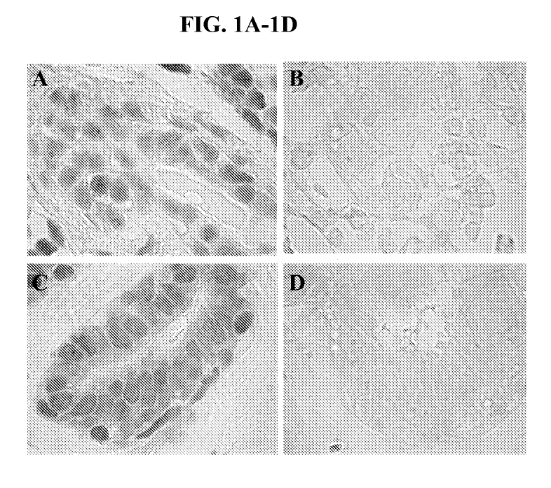

In this particular example, all three of the BRCA2 hereditary breast cancer cases show truncation. FIG. 1 illustrates results of IHC with the BRCA2 C-terminal antibody.

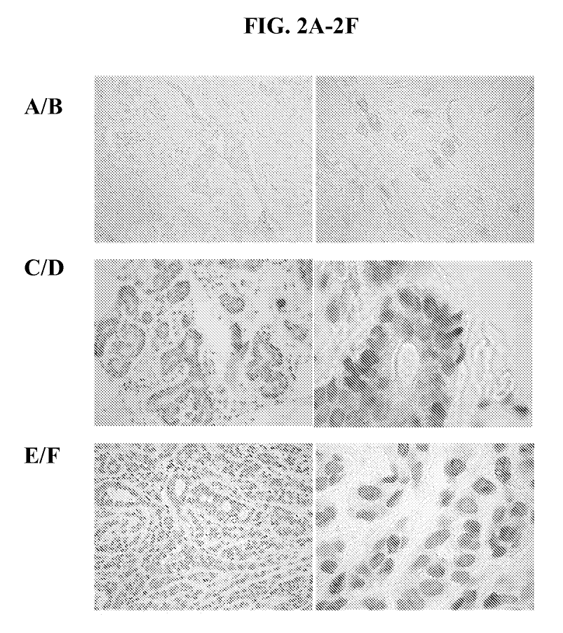

One exemplary method displayed in FIG. 1 demonstrates an IHC performed on human breast tissue with C-terminal BRCA2 antibody. Left panels use the C-terminal BRCA2 antibody as the primary antibody and the right panels use immunostained instead with IgG and serve as a negative control. The dark grey represents specific staining of the antibody peroxidase reaction and the light grey color represents a neutral counter stain MetanilYellow. All panels are 100× magnification. Tissue sections show normal breast (upper panels), and sporadic cancer (right panels). Note BRCA2 peptide blocks most immunostaining. FIG. 1 illustrates the C-terminal BRCA2 antibody appears specific for the nuclear BRCA2 protein. FIG. 2 illustrates an exemplary result of C-terminal BRCA2 antibody immunostaining on hereditary breast cancers.

One exemplary method displayed in FIG. 2 demonstrates an IHC performed on human breast tissue using the C-terminal BRCA2 antibody. The dark grey represents specific staining of the antibody peroxidase reaction and the light grey color represents a neutral counter stain Metanil Yellow. Left panels are 20× and right panels 100× magnification. Tissue sections show BRCA2 Hereditary Cancer (upper panels); normal breast from BRCA2 Hereditary Cancer patient (middle panels), sporadic cancer (lower panels). Note the absence of staining in the hereditary cancer sections (upper right and left panels). BRCA2 protein truncation, due to the 6174delT truncation mutant in this patient also demonstrates the specificity of this antibody in tissue. Twenty sporadic cancers were stained and all show moderate to strong staining with C-terminal BRCA2 as in the lower panels.

### N-Terminal IHC on Human Breast Tissue:

In order to characterize the BRCA2 N-terminal antibody for IHC its specificity was tested on normal human breast tissue. One exemplary method displayed in FIG. 2 demonstrates an IHC performed on normal breast using C-terminal antibody (left panel), N-terminal antibody (middle panel), and IgG serving as a negative control. The right panels are blank because the specific antibody is not present so no filled in circles (nuclei) are stained. The left panels show filled in circles (nuclei) which are stained red (dark grey in figure) by the C-terminal antibody.

FIG. 2 illustrates that the two upper panels are blank likely due to the hereditary cancer cells lacking the end of the protein so no staining is present with the C-terminal antibody. The bottom four panels show filled in circles (nuclei) which are stained red (dark grey in figure) by the C-terminal antibody.

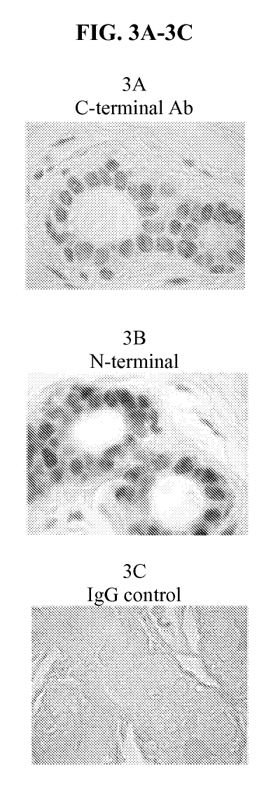

In one exemplary method, FIG. 3 illustrates three panels of tissue samples analyzed by IHC techniques. The filled in circles (nuclei) are stained red (dark grey in figure) (left panel), black (middle panel), and unstained in the negative control panel (right panel).

FIG. 1 represents exemplary IHC where the right panels are blank because the specific antibody is not present so no filled in circles (nuclei) are stained. The left panels show filled in circles (nuclei) which are stained red (dark grey in figure) by the C-terminal antibody.

FIG. 2 represents exemplary IHC where the two upper panels are blank because the hereditary cancer cells lack the end of the protein so no staining is present with the C-terminal antibody. The bottom four panels show filled in circles (nuclei) which are stained red (dark grey in figure) by the C-terminal antibody.

FIG. 3: The filled in circles (nuclei) are stained red (dark grey in figure) (left panel), black (middle) panel, and unstained in the negative control panel (right panel).

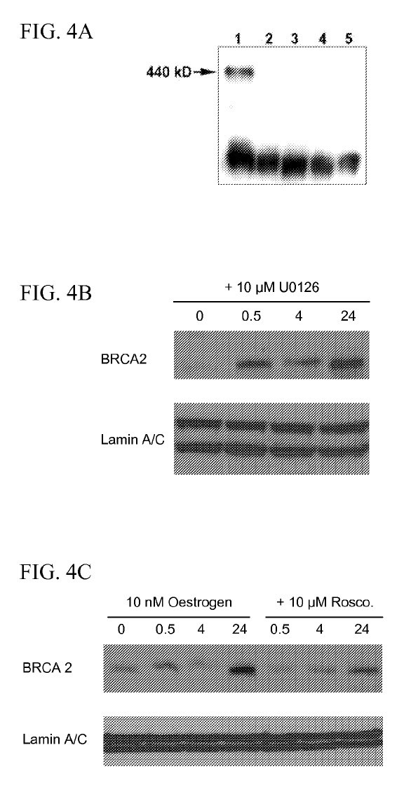

FIG. 4 illustrates an exemplary Western blot. Western Blot with S3291 BRCA2 phosphospecific Ab on BRCA2-defective V-C8 Lane 1: V-C8+human BRCA2, Lane 2: V-C8 +human BRCA2 treated with 10 Gy Lane 3: V-C8 Lane 4: V-C8 treated with 10 Gy. Lane 5: MCF-7 cells. 440 kD band is full-length human BRCA2.

### Example 2

Estrogen mediated phosphorylation and non-genomic stabilization of BRCA2 protein in breast cells.

In one exemplary method, blood-inherited mutations were analyzed in human BRCA2. These gene mutations often cause cancer in hormonally responsive tissues including breast and ovary and the resulting tumors. In addition these tissues are frequently oestrogen receptor positive. Oestrogen affects cells via its receptor through transcriptional effects and through non-genomic effects on cellular signals for example through protein kinases. It was demonstrated that oestrogen rapidly phosphorylates BRCA2 at serine 3291 leading to a cdk-2 dependent protein stabilization in oestrogen regulated cell lines. This is a non-genomic effect which is unaffected by protein synthesis inhibitors but is blocked by tamoxifen and ICI 182,780. Oestrogen physiologically regulates BRCA2 expression because BRCA2 is highly expressed and phosphorylated in normal breast having oestrogen receptor positive breast cancer but not in oestrogen receptor negative breast cancers. In keeping with its cdk2-mediated stabilization of BRCA2, oestrogen enhances survival following irradiation but this effect is blocked by the cdk2 inhibitor roscovitine. Studies of human breast tissue show that normal breast glands express phosphorylated BRCA2 in nuclei and that BRCA2 is phosphorylated and abundant in ten ER (oestrogen receptor) positive cancers but absent in ten ER negative cancers, analogous to the results of oestrogen treated cultured cells. These results demonstrate how oestrogen may function in DNA repair and provides a link between hormone action and BRCA gene function in studies of breast cancer. Furthermore it provides an explanation for the breast/ovarian specificity of the BRCA2 gene mutations. These results indicate that one therapeutic approach to treatment of these cancers should design therapeutics that link oestrogen blockade with other agents for example, agents that target specific DNA repair pathways.

The majority of breast tumors from patients with germline mutations in the BRCA2 gene express oestrogen receptor. This correlation suggests a connection between oestrogen action and BRCA2 tumor suppression. One possibility is that oestrogen positively regulates the levels or the activity of BRCA2. Oestrogen may influence post-translational modification of BRCA2 such as the cdk2 phosphorylation previously described or through non-genomic actions to exert this regulation. Therefore, in another exemplary method the effect of oestrogen treatment on BRCA2 in hormone-responsive MCF7 and T47D human breast cancer cells was examined.

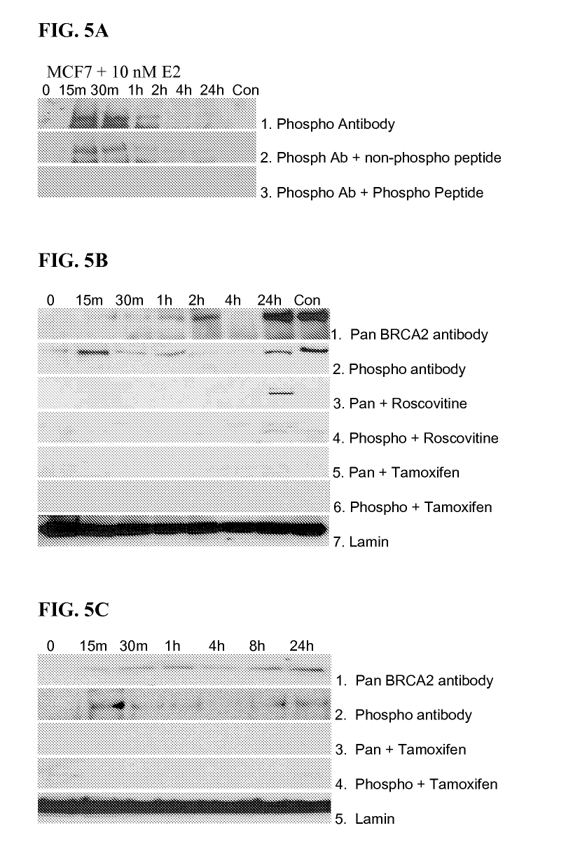

In one example, the MCF7 and T47D (MCF-7: Michigan Cancer Foundation line #7; T47D: a 47^{th} tumor line and had ductal carcinoma origin.) were used. The cells were cultured in charcoal stripped/phenol free medium for two days prior to treatment with 10 nM oestrogen for early (0.5 h), intermediate (4 h), or extended (24 h) times. MCF7 (FIG. 5A) and T47D (FIG. 5C) cells both exhibited rapid increases in BRCA2 protein levels, suggesting a non-genomic mechanism involving protein stabilization as a possibility. Indeed, increased protein levels were still observed at early and intermediate time points in cells pre-treated with 25 μM cycloheximide to inhibit protein synthesis (FIG. 5A). In one example, Cdk-2 phosphorylates BRCA2 at S3291, therefore cells were pretreated with the cyclin-dependent kinase inhibitor roscovitine which repressed the early increase of BRCA2 (FIG. 5A, 5C). In contrast, inhibition of the MAPK pathway, a common mediator or non-genomic oestrogen effects, had no effect on the rapid increase of BRCA2 levels (FIG. 5B).

Following the observation that treatment with roscovitine inhibits the initial effect of oestrogen treatment on BRCA2, previously characterized phosphorylation at serine 3291 in BRCA2 was investigated for its participation in the effects of oestrogen. In order to examine BRCA2 phosphorylation at S329 1, a phosphospecific antisera was generated by immunizing rabbits with the phosphorylated peptide TFVSPAAKAGG (SEQ ID NO:1) and then affinity purified by selective elution with phosphorylated and unphosphorylated peptide bound to sepharose. As a stringent test of antibody specificity, peptide blocks with the phosphorylated and the unphosphorylated peptide of sequence TFVSPAAQKAGG (SEQ ID NO:1) were analyzed and showed that the phosphorylated BRCA2 protein was appropriately blocked by phosphorylated but not by unphosphorylated peptides (FIG. 5A).

Then S3291 phosphorylated BRCA2 was analyzed and 3291 serine phosphorylation was increased following oestrogen treatment. In fact, the oestrogen effect on serine 3291 phosphorylation occurred within 15-30 minutes of treatment (FIG. 5B). In order to determine whether the estrogen receptor mediated this effect, whether the antioestrogen, 4-hydroxytamoxifen could eradicate the increase in BRCA2 serine 3291 expression following oestrogen treatment was investigated. Pretreatment with 4-hydroxytamoxifen blocked the estrogen mediated increase in both BRCA2 protein phosphorylation and stabilization in MCF-7 cells (FIG. 5B) and T47D cells (FIG. 5C). Pretreatment with the absolute estrogen antagonist ICI 182,780 also blocks the estrogen mediated increase in BRCA2 S3291 phosphorylation and protein levels (data not shown).

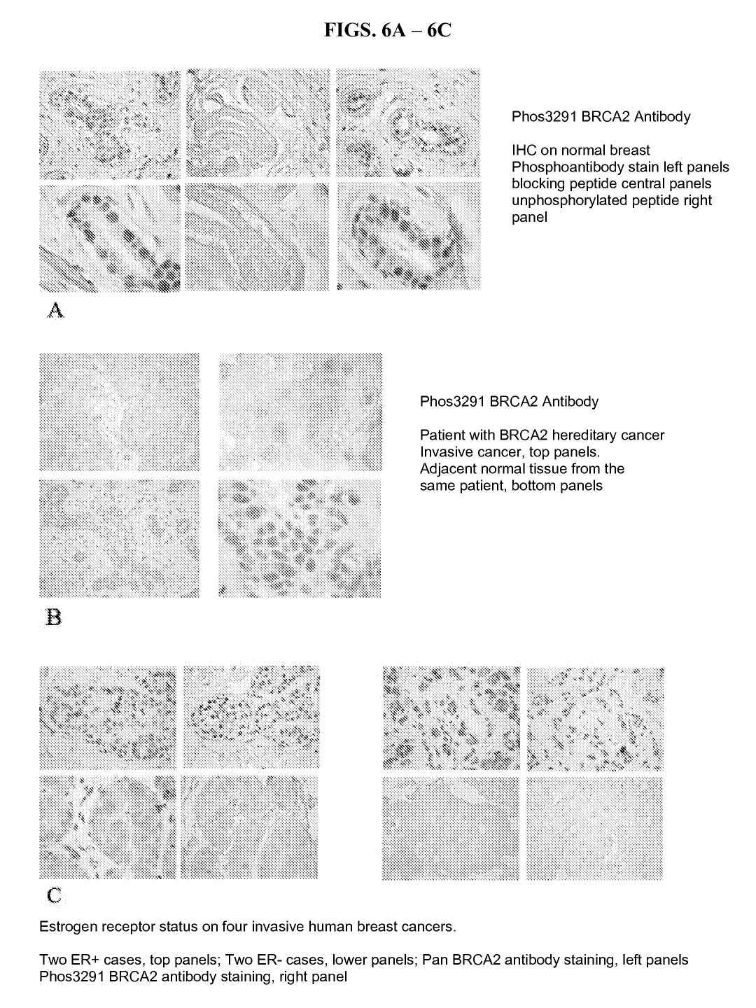

In one exemplary method, because breast cell lines often poorly mimic expression in breast tissues and cancers, BRCA2 expression and S3291 phosphorylation in breast tissue samples using immunohistochemistry was examined. The phosphospecific antibody to S3291 BRCA2 clearly stains nuclei within a normal breast gland and this result is blocked by phosphopeptide (FIG. 6A middle panel) but not unphosphorylated peptide (FIG. 6A right panel), providing evidence for specificity of this reagent. As an additional test for antibody specificity, breast cancer samples from patients from BRCA2 hereditary cancer (6174delT mutation) were tested and showed a loss of nuclear immunostaining in the cancers but not the normal tissues from these patients. Because these patients have a truncated BRCA2 protein which lacks this S3291 phosphorylation site in the cancers but retain the wild type allele in normal tissue, this was anticipated. Having characterized the antibody both biochemically (peptide block) and genetically (genetic cancer) whether there is a correlation between estrogen receptor and S3291 BRCA2 phosphorylation in sporadic breast cancers was analyzed. The 10 ER+ cancers had strong S3291 BRCA2 phosphorylation and the 10ER negative cancers had weak or absent S3291 BRCA2 phosphorylation (FIG. 6C). Representative IHC demonstrate a loss of staining for both phosphorylated and total BRCA2 protein IHC in human ER negative breast cancers. These results indicate that BRCA2 protein expression and phosphorylation are similarly regulated in human breast cancers, see for example, the ER+breast cancer cell lines MCF7 and T47D.

In another exemplary method, having demonstrated that the oestrogen regulation of BRCA2 in both human mammary cancer cell lines (MCF7 and T47D) and human cancers, the oestrogen effects on BRCA2 function following radiation treatment were investigated in order to determine its biological significance. In one exemplary method, estrogen treatment was shown to increase the number of MCF7 cells which survive 1, 2 or 4 Gy of ionizing radiation but that the cdk-2 inhibitor purvalanol A inhibits much of the increased radiation survival attributed to estrogen (data not shown). These results indicate that oestrogen effects radiation response and likely DNA repair properties in part through cdk2 phosphorylation and stabilization of BRCA2 protein.

In another exemplary method, an IHC was performed on normal human breast tissue using an exemplary Phos3291 BRCA2 Antibody. Sections were taken that represent adjacent serial sections from a tissue block. Using these sections it was demonstrated that phosphoantibody staining was completely blocked by phosphorylated peptide TFVSPAAQKAGG (SEQ ID NO:1), but not by unphosphorylated peptide. An IHC was performed on human breast tissue using the Phos3291 BRCA2 Antibody. All tissues were from a patient with BRCA2 hereditary cancer with 6174delT mutation. Tissue sections were invasive cancer and adjacent normal tissue. Because the patients normal tissue expresses both wild type and mutant alleles there is expression certain sections but not in the cancer section in which only the truncated 6174delT mutant allele is expressed. An IHC was performed on sporadic breast cancers, ER+ and ERneg. Certain sections were exposed to panBRCA2 antibody and others exposed to phosphospecific BRCA2 antibody. Note phosphospecific BRCA2 protein is absent in ERneg cancers and total BRCA2 protein is low.

### Example 3

Visualization of Truncated BRCA2 proteins in Hereditary Breast Cancer (Blinded Feasibility Study)

A pilot study was used to determine whether immunohistochemistry (IHC) using C-terminal and N-terminal antibodies could be used to distinguish BRCA2 hereditary cancers from sporadic breast cancers. In one exemplary method, a study group included 3 patients with BRCA2 hereditary breast cancer, 3 patients with BRCA1 hereditary breast cancer, and 20 patients with sporadic breast cancer. In this example, this study was conducted in a blinded manner so the technician performing the staining and the pathologists reading the slides were unaware which samples were sporadic and which were inherited cancers. Tissue sections were cut from paraffin blocks and heat inactivated in a decloaking chamber for 5 minutes in sodium citrate buffer pH 6.0 to allow antigen retrieval. Blinded slides were then quantitated by a combined scoring scale which assesses both immunostaining intensity and percent positive cells. Intensity was rated from 0-3 and percent positive cells was rated from 0-3 and these two scores were added and then results were decoded and unblinded to compare with known BRCA2 mutation status (see Table 2)

TABLE 2
BRCA2 Truncation Ratio in Hereditary Cancers and Controls

| | Mean | | Mean Truncation |
|---|---|---|---|
| | N-terminal | Mean C-terminal | Ratio |
| Cancer Type | IHC score | IHC score | (C score/N score) |
| BRCA 2 Hereditary | 5.1 | 0 | 0 |
| BRCA 1 Hereditary | 5.3 | 5.7 | 1.08 (range 1-1.2) |
| Sporadic cancer | 4.8 | 5.5 | 1.15 (range 0.7-1.5) |

The BRCA2 hereditary breast cancer cases show truncation since the C score/N score was zero. None of the BRCA1 cases or sporadic cancer cases were misclassified because all had truncation ratios greater than 0.7.

### Development of Monoclonal Antibody for BRCA2 C-Terminus:

In one exemplary method, a monoclonal antibody was generated from the C-terminal BRCA2 antibody to the peptide region 3284-3294. As an initial test of this reagent immunized mice were screen by an ELISA assay and then tested by high titer mouse sera by an initial IHC on cell pellets, followed by western blotting, then IHC on two BRCA2 hereditary cancer samples. Mice were then chosen for splenectomy and cell fusion based on IHC specificity for C-terminal BRCA2 protein. Clonal supernatants from fusions were then similarly screened by ELISA, western blot, and IHC. Clone 575A15 was selected and subcloned.

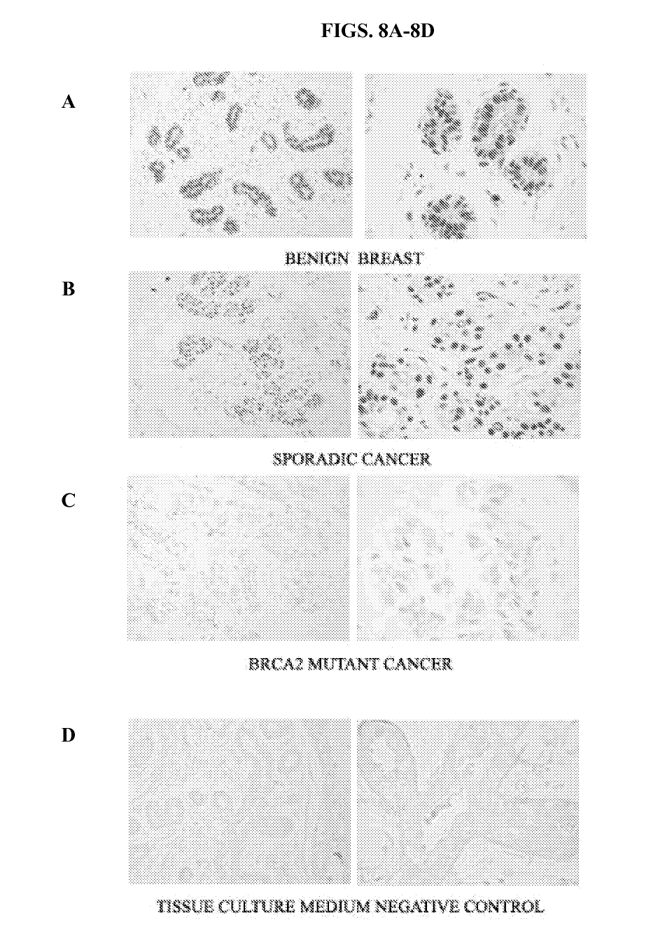

FIGS. 8A to 8D represent IHC performed on human breast tissue with C-terminal BRCA2 monoclonal antibody 575A15. Upper panels (FIG. 8A) are normal breast tissue, upper middle panels are from a sporadic ER+ breast cancer (FIG. 8B), lower middle panels are from a BRCA2 hereditary cancer (FIG. 8C), and the lowest panel is a negative control using a monoclonal expansion media supernatant in place of the specific antibody (FIG. 8D). Magnifications are 20× (left panels) and 60× (right panels) for each row. Each experiment used horseradish peroxidase which stains brown and a counterstain that stains metanil yellow.

In another exemplary experiment, tissues were exposed to a corresponding rabbit polyclonal antibody directed against BRCA2 C-terminal amino acids 3284-3294. The polyclonal antisera used in this exemplary method can be used as a diagnostic backup for ambiguous cases. For example, if mouse monoclonal and a rabbit polyclonal antisera have differing artifactual staining. There was an absence of immunostaining in the Hereditary cancer sections due to BRCA2 protein truncation. IHC was performed on human breast tissue using the polyclonal rabbit C-terminal BRCA2 antibody. The antibody peroxidase reaction stains brown and the neutral counter stain stains Metanil Yellow. Tissue sections illustrated normal breast, sporadic breast cancer, and BRCA2 Hereditary Cancer.

### Using Phosphospecific Monoclonal Antibodies to Predict PARP Inhibitor Response:

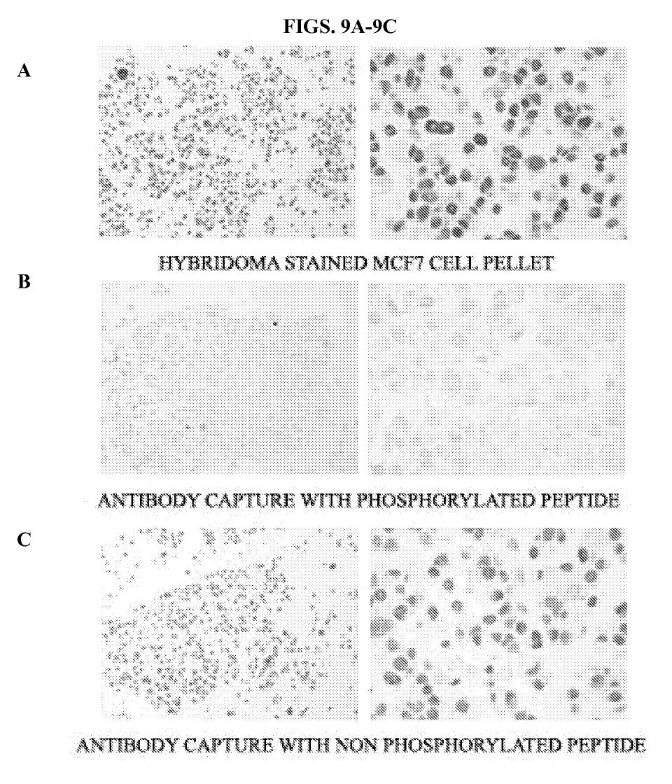

In certain exemplary methods, monoclonal antibodies were developed specific for BRCA2 phosphorylated at Serine 3291. Initially, these antibodies were characterized by ELISA titer to the phosphorylated versus unphosphorylated peptides and by western blotting, but also by IHC as a means to select a monoclonal for IHC. FIG. 9A-9C represents that the BRCA2 monoclonal 576A12-TGI is specific for phosphorylated BRCA2 because incubation of the antibody with phosphorylated peptide coupled to sepharose eliminates the signal whereas incubation of the antibody with unphosphorylated peptide coupled to sepharose does not decrease the signal.

FIGS. 9A-9C represent an exemplary experiment of MCF7 cells grown on cover slips and treated with 10% serum and immunostained with BRCA2 phospho-specific monoclonal 576A12-TGI. The left panels are 20× magnification and right panels are 60×. Middle panels show results of antibody captured with 1/10th volume of phosphopeptide sepharose; and lower panels show results of antibody captured with 1/10th volume of unphosphorylated peptide-sepharose. The antibody capture consisted of a 1 hour room temperature incubation followed by centrifugation (1 minute 5000 rpm) to remove antibody bound to sepharose. The sequence of the phosphorylated peptide: CTFV[phosphoS] AAQK and the unphosphorylated peptide:

CTFVSAAQK.

Having demonstrated the phosphospecificity of the monoclonal antibody with cultured cells, IHC was performed on formalin fixed, paraffin-embedded tissue sections from normal breast, breast cancer and BRCA2 mutant cancer (which should have no immunostaining because this region of the protein is deleted via the truncating mutation).

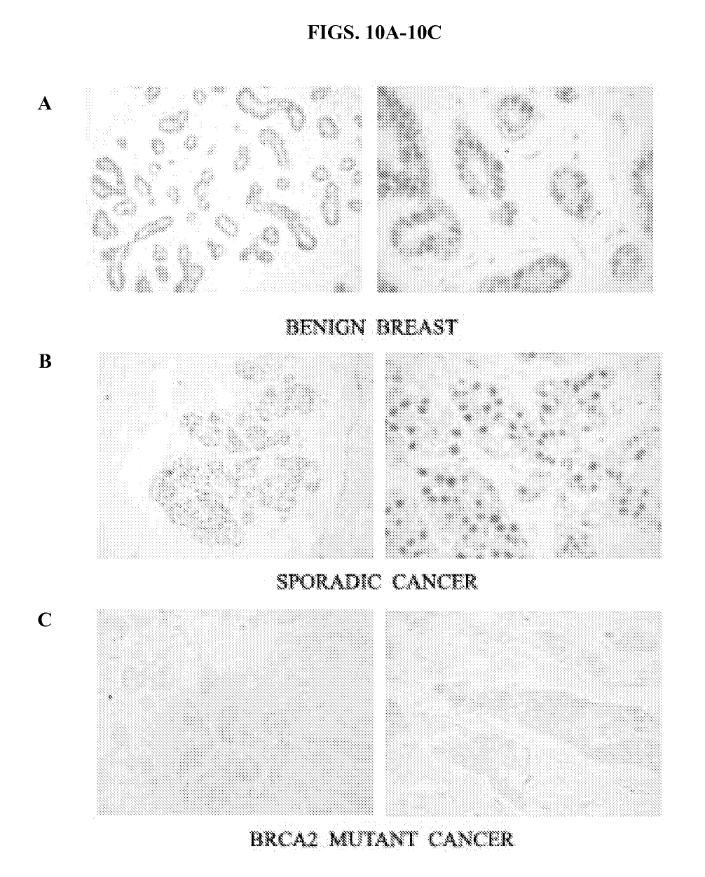

In one exemplary method, IHC with BRCA2 phospho-specific monoclonal 576A12-TGI was demonstrated (see FIGS. 10A-10C). The left panels are 20× magnification and the right panels are 60×. The upper panels are normal human breast tissue; middle panels are sporadic human breast cancer; and the lower panels are from a BRCA2 mutant breast cancer which should be negative (and represent no binding) because the C-terminal portion of BRCA2 where this phosphorylation site is present is absent from this mutant protein.

Identifying sporadic cancers that lack BRCA2 phosphorylation but have intact C-terminal BRCA2 protein in ovarian cancer: In one exemplary experiment, immunostaining with the monoclonal 575CA15 was presented in FIG. 8A-8D. As demonstrated for normal breast tissue and breast cancer, BRCA2 C-terminal monoclonal antibody stains the nuclei of normal ovarian surface epithelium and stains the nuclei of sporadic (non-hereditary) cancer.

Another exemplary experiment, however, illustrates a phosphorylation-specific monoclonal antibody, 575A12, immunostains normal ovarian epithelium and some ovarian cancers but does not stain other ovarian cancers even though the same sections stains with the C-terminal monoclonal antibody previously described.

In another example, cancer nuclei are not stained with the phosphospecific monoclonal although the same nuclei are stained with the C-terminal antibody (which stains phosphorylated and non-phosphorylated proteins equally). This provides an example of an ovarian cancer with BRCA2 protein which is nonphosphorylated and therefore likely to respond to PARP inhibitors.

### Example 4

Mutations in the BRCA1 gene are thought to account for roughly 45% of inherited breast cancer and 80-90% of families with increased risk of early onset breast and ovarian cancer. In certain exemplary methods, antibodies (e.g. monoclonal antibodies) will be developed specific for detection of BRCA1 truncations and/or post-translational modifications. Initially, these antibodies will be characterized by ELISA titer to the truncated versus wild type peptides and by western blotting, but also by IHC as a means to select for example, a monoclonal for IHC. In other exemplary methods, specific antibodies that detect post-translational modifications in the BRCA1 gene will also be identified. In certain exemplary methods, changes in the phosphorylation of BRCA1 proteins will be identified in cancerous versus non-cancerous tissues. Then, antibodies will be generated to predetermined phosphorylated amino acids. Certain exemplary phospho-specific antibodies will be used to detect the presence or absence of pre-determined phosphorylated amino acids of BRCA1 in a tissue sample from a subject. In other exemplary methods, antibodies identified for detection of truncated BRCA1 protein or specific phosphorylated amino acids can be used to predict the onset or presence of breast and/or ovarian cancer in a subject. In addition, these antibodies will also be used to predict the response of a subject to potential therapeutic treatments for breast or ovarian cancers.

### Example 5

### Tissue Truncation Test for Muscular Dystrophy

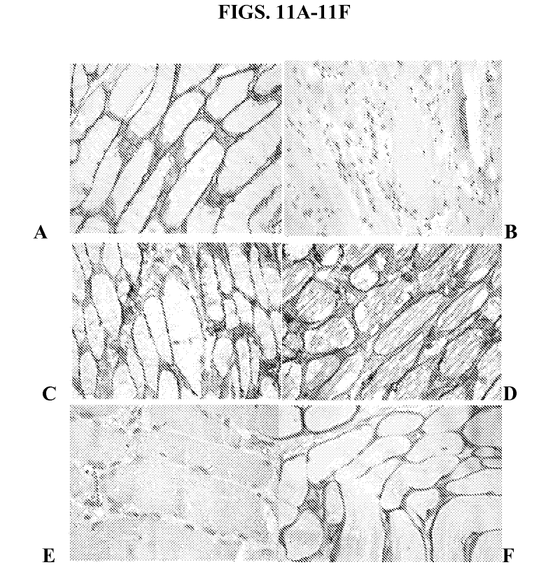

In another exemplary method, the Tissue-based Protein Truncation Test for detection of the truncating mutation responsible for muscular dystrophy was examined. FIG. 11A-11F represent results of IHC with a C-terminal antibody for dystrophin which shows a lack of staining in a Duchenne's patient with a truncating mutation in exon 35 (FIG. 11E, lower left panel) but membrane staining in other muscle diseases

In one exemplary method, IHC of skeletal muscle biopsies using a C-terminal dystrophin antibody were demonstrated. Darker staining (typically brown due to the peroxidase staining) illustrates dystrophin protein at cell membrane with nuclei counterstainedwith hematoxylin (see light grey spotted areas which are blue when photps are in color). All photos are 20× magnification. Samples include normal skeletal muscle (FIG. 11A, upper left panel); negative control skeletal muscle with IgG but no dystrophin antibody (FIG. 11B, upper right); merosin deficiency (FIG. 16C, middle left); Pompe's disease (FIG. 11D, middle right); Duchenne's Muscular Dystrophy (FIG. 16E, lower left); Becker's Dystrophy (FIG. 11F, lower right). Note that the biopsy of the Duchenne's patient with the truncating mutation in Dystrophin (lower left) is the only non-control sample which lacks cell surface staining.

TABLE 3
Chemical Formulas and Names for PARP inhibitors:

| | |
|---|---|
| 1. | Name: BGP-15 |
| | Chemical Name: O-(3-piperidino-2-hydroxy-1-propyl)nicotinic |
| | amidoxime |
| | Chemical Formula: C₁₄H₁₉N₄O₂ |
| 2. | Name: DPQ |
| | Chemical Name: 3,4-Dihydro-5[4-(1-piperindinyl)butoxy]-1(2H)- |
| | isoquinoline |
| | Chemical Formula: C₁₈H₂₆N₂O₂ |
| 3. | Name: INH₂BP |
| | Chemical Name: 5-Iodo-6-amino-1,2-benzopyrone |
| | Chemical Formula: C₉H₆INO₂ |
| 4. | Name: IQD |
| | Chemical Name: 1,5-Dihydroxyisoquinoline |
| | Chemical Formula: C₉H₇NO₂ |
| 5. | Name: EB47 |
| | Chemical Name: 5-Hydroxy-1(2H)-isoquinoline 1,5-Isoquinolinediol |
| | Chemical Formula: C₂₄H₂₇N₉O₆•2HCl•2H₂O |
| 6. | Name: 4-ANI |
| | Chemical Name: 4-amino-1,8,-napthalamide |
| | Chemical Formula: C₁₂H₈N₂O₂ |
| 7. | Name: NU1025 |
| | Chemical Name: 8-Hydroxy-2-methylquinazoline-4-one |
| | Chemical Formula: C₉H₈N₂O₂ |
| 8. | Name: 5-AIQ |
| | Chemical Name: 5-aminoisoquinolinone |
| | Chemical Formula: C₉H₈N₂O |
| | Chemical Structure: |
| 9. | Name: PJ34 |
| | Chemical Name: N-(6-oxo-5,6-dihydro-phenanthridin-2-yl)-N,N- |
| | dimethylacetamide |
| | Chemical Formula: C₁₇H₁₇N₃O₂•HCl |
| | Chemical Structure: |
| 10. | Name: TIQ-A |
| | Chemical Name: 4H-Thieno[2,3-c]isoquinolin-5-one |
| | Chemical Formula: C₁₁H₇NOS |
| | Chemical Structure: |
| 11. | Name: AG-014361 |
| | Chemical Structure: |

In addition: some basic structures for generic chemical structure claims, if
possible:
General Structure of a PARP inhibitor:

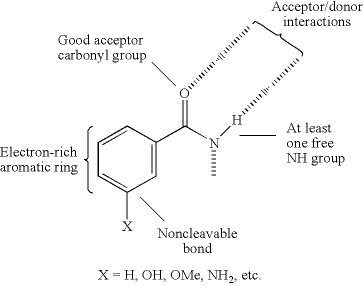

Structural requirements for potent PARP inhibitors
Structure of a tricyclic benzimidazole compound:

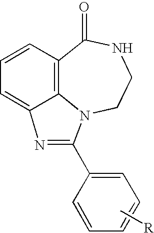

Structure of a tricyclic lactam indole compound:

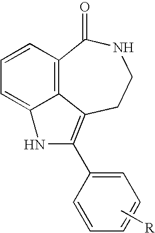

TABLE 4
1: SEVERE COMBINED IMMUNODEFICIENCY, AUTOSOMAL
RECESSIVE, WITH CRAC CHANNEL DYSFUNCTION, INCLUDED
2: FANCM GENE; FANCM
FANCONI ANEMIA, COMPLEMENTATION GROUP M, INCLUDED; FANCM, INCLUDED
Gene map locus 14q21.3
3: ICHTHYIN
CONGENITAL ICHTHYOSIS, AUTOSOMAL RECESSIVE, INCLUDED
Gene map locus 5q33
4: FRAGILE SITE, FOLIC ACID-TYPE, RARE,
FRA(10)(q23.3), CANDIDATE GENE 1
5: PEROXISOME BIOGENESIS FACTOR 26; PEX26
PEROXISOME BIOGENESIS DISORDER, COMPLEMENTATION GROUP 8, INCLUDED
Gene map locus 22q11.21
6: PHD FINGER PROTEIN 9; PHF9
FANCONI ANEMIA, COMPLEMENTATION GROUP L, INCLUDED; FANCL, INCLUDED
Gene map locus 2p16.1
7: NPC1-LIKE 1; NPC1L1
EZETIMIBE, NONRESPONSE TO, INCLUDED
Gene map locus 7p13
8: SEMAPHORIN 7A; SEMA7A
JMH BLOOD GROUP, INCLUDED
Gene map locus 15q22.2-q23
9: GLUTAMATE-CYSTEINE LIGASE, CATALYTIC SUBUNIT; GCLC
MYOCARDIAL INFARCTION, SUSCEPTIBILITY TO, INCLUDED
Gene map locus 6p12
10: LIM DOMAIN-BINDING 3; LDB3
CARDIOMYOPATHY, DILATED, WITH LEFT VENTRICULAR NONCOMPACTION, INCLUDED
Gene map locus 10q22.2-q23.3
11: CLAUDIN 14; CLDN14
DEAFNESS, AUTOSOMAL RECESSIVE 29, INCLUDED; DFNB29, INCLUDED
Gene map locus 21q22.3
12: RETINITIS PIGMENTOSA GTPase
REGULATOR-INTERACTING PROTEIN; RPGRIP1 LEBER CONGENITAL AMAUROSIS, TYPE VI, INCLUDED; LCA6,
INCLUDED
Gene map locus 14q11
13: ADIPOCYTE, C1Q, AND COLLAGEN DOMAIN CONTAINING; ACDC
ADIPONECTIN DEFICIENCY, INCLUDED
Gene map locus 3q27
14: PROTEIN Z-DEPENDENT PROTEASE INHIBITOR PRECURSOR
VENOUS THROMBOSIS, SUSCEPTIBILITY TO, INCLUDED
Gene map locus 14q32.1
15: MANNAN-BINDING LECTIN SERINE PROTEASE 2; MASP2
MAP19, INCLUDED
Gene map locus 1p36.3-p36.2
16: PRESTIN; PRES DEAFNESS, NEUROSENSORY, AUTOSOMAL RECESSIVE, 61,
INCLUDED; DFNB61, INCLUDED
Gene map locus 7q22.1
17: KLOTHO; KL
CORONARY ARTERY DISEASE, SUSCEPTIBILITY TO, INCLUDED
Gene map locus 13q12
18: MER TYROSINE KINASE PROTOONCOGENE; MERTK
RETINITIS PIGMENTOSA, MERTK-RELATED, INCLUDED
Gene map locus 2q14.1
19: CD209 ANTIGEN; CD209
DENGUE FEVER, PROTECTION AGAINST, INCLUDED
Gene map locus 19p13.3
20. B-CELL LINKER PROTEIN; BLNK
HYPOGLOBULINEMIA AND ABSENT B CELLS, INCLUDED
Gene map locus 10q23.2
21: VOLTAGE-DEPENDENT ANION CHANNEL 1; VDAC1
VDAC DEFICIENCY, INCLUDED
Gene map locus 5q31
22: ALPHA-METHYLACYL-CoA RACEMASE; AMACR
ALPHA-METHYLACYL-CoA RACEMASE DEFICIENCY, INCLUDED
Gene map locus 5p13.2-q11.1
23: CHECKPOINT KINASE 2, S. POMBE, HOMOLOG OF; CHEK2
BREAST AND COLORECTAL CANCER, SUSCEPTIBILITY TO, INCLUDED
Gene map locus 22q12.1
24: PROMININ 1; PROM1
RETINAL DEGENERATION, AUTOSOMAL RECESSIVE, PROMININ-RELATED, INCLUDED
Gene map locus 4p16.2-p12
25: CRUMBS, DROSOPHILA, HOMOLOG OF, 1; CRB1
LEBER CONGENITAL AMAUROSIS DUE TO DEFECT IN CRB1, INCLUDED
Gene map locus 1q31-q32.1
26: SODIUM CHANNEL, VOLTAGE-GATED, TYPE IV, ALPHA
SUBUNIT; SCN4A MYASTHENIC SYNDROME DUE TO MUTATION IN SCN4A, INCLUDED
Gene map locus 17q23.1-q25.3
27: POTASSIUM CHANNEL, VOLTAGE-GATED, ISK-RELATED SUBFAMILY, MEMBER 2;
KCNE2 LONG QT SYNDROME 6, INCLUDED; LQT6, INCLUDED
Gene map locus 21q22.1
28: CYTOCHROME P450, FAMILY 7, SUBFAMILY B, POLYPEPTIDE 1; CYP7B1
BILE ACID SYNTHESIS DEFECT, CONGENITAL, 3, INCLUDED
Gene map locus 8q21.3
29: SPINOCEREBELLAR ATAXIA 10; SCA10
ATAXIN 10, INCLUDED; ATXN10, INCLUDED
Gene map locus 22q13
30: FANCONI ANEMIA, COMPLEMENTATION GROUP F; FANCF
FANCF GENE
Gene map locus 11p15
31: THYROID-STIMULATING HORMONE RECEPTOR; TSHR
THYROID ADENOMA, HYPERFUNCTIONING, INCLUDED
Gene map locus 14q31
32: PEROXISOME BIOGENESIS FACTOR 16; PEX16
PEROXISOME BIOGENESIS DISORDER, COMPLEMENTATION GROUP 9, INCLUDED
Gene map locus 11p12-p11.2
33. GAP JUNCTION PROTEIN, BETA-3; GJB3
ERYTUROKERATODERMIA VARIABILIS, AUTOSOMAL RECESSIVE, INCLUDED
Gene map locus 1p35.1
34: JERKY, MOUSE, HOMOLOG OF; JRK EPILEPSY, CHILDHOOD ABSENCE, EVOLVING TO
JUVENILE MYOCLONIC EPILEPSY, INCLUDED
Gene map locus 8q24
35: METHYLMALONATE SEMIALDEHYDE DEHYDROGENASE; MMSDH
METHYLMALONATE SEMIALDEHYDE DEHYDROGENASE DEFICIENCY, INCLUDED
Gene map locus 14q24.3
36: PEROXISOME BIOGENESIS FACTOR 3; PEX3
PEROXISOME BIOGENESIS DISORDER, COMPLEMENTATION GROUP 12, INCLUDED
Gene map locus 6q23-q24
37: TOLL-LIKE RECEPTOR 4; TLR4
ENDOTOXIN HYPORESPONSIVENESS, INCLUDED
Gene map locus 9q32-q33
38: 3-PRIME-alphaPHOSPHOADENOSINE 5-PRIME-PHOSPHOSULFATE SYNTHASE 2; PAPSS2
SPONDYLOEPIMETAPHYSEAL DYSPLASIA, PAKISTANI TYPE, INCLUDED
Gene map locus 10q22-q24
39: X-RAY REPAIR, COMPLEMENTING DEFECTIVE, IN CHINESE HAMSTER, 9; XRCC9
FANCONI ANEMIA, COMPLEMENTATION GROUP G, INCLUDED; FANCG, INCLUDED
Gene map locus 9p13
40: FATTY ACID AMIDE HYDROLASE; FAAH
DRUG ADDICTION, SUSCEPTIBILITY TO, INCLUDED
Gene map locus 1p35-p34
41: AQUAPORIN 9; AQP9
GLYCEROL RELEASE DURING EXERCISE, DEFECTIVE, INCLUDED
Gene map locus 15q22
42: PEROXISOME BIOGENESIS FACTOR 10; PEX10
PEROXISOME BIOGENESIS DISORDER, COMPLEMENTATION GROUP 7, INCLUDED
Gene map locus Chr.1
43: ACTIVIN A RECEPTOR, TYPE IIB; ACVR2B
LEFT-RIGHT AXIS MALFORMATIONS, INCLUDED
Gene map locus 3p22-p21.3
44: PAIRED-LIKE HOMEODOMAIN TRANSCRIPTION FACTOR 3; PITX3
CATARACT, CONGENITAL, INCLUDED
Gene map locus 10q25
45: LOW DENSITY LIPOPROTEIN, OXIDIZED, RECEPTOR 1; OLR1
MYOCARDIAL INFARCTION, SUSCEPTIBILITY TO, INCLUDED
Gene map locus 12p13-p12
46: FORKHEAD BOX C2; FOXC2 LYMPHEDEMA-DISTICHIASIS SYNDROME WITH RENAL DISEASE AND DIABETE [symbol]
MELLITUS, INCLUDED
Gene map locus 16q24.3
47: L-ARGININE: GLYCINE AMIDINOTRANSFERASE; GATM
ARGININE: GLYCINE AMIDINOTRANSFERASE DEFICIENCY, INCLUDED
Gene map locus 15q15.3
48: CONE-ROD HOMEOBOX-CONTAINING GENE; CRX
LEBER CONGENITAL AMAUROSIS DUE TO DEFECT IN CRX, INCLUDED
Gene map locus 19q13.3
49: PEROXISOME BIOGENESIS FACTOR 1; PEX1
PEROXISOME BIOGENESIS DISORDER, COMPLEMENTATION GROUP 1, INCLUDED
Gene map locus 7q21-q22
50: CALCIUM CHANNEL, VOLTAGE-DEPENDENT, BETA-4 SUBUNIT; CACNB4
EPISODIC ATAXIA, TYPE 5, INCLUDED; EA5, INCLUDED
Gene map locus 2q22-q23
51: JAGGED 1; JAG1
DEAFNESS, CONGENITAL HEART DEFECTS, AND POSTERIOR EMBRYOTOXON, INCLUDED
Gene map locus 20p12
52: RETINA AND ANTERIOR NEURAL FOLD HOMEOBOX GENE; RAX
ANOPHTHALMIA/MICROPHTHALMIA, AUTOSOMAL RECESSIVE, INCLUDED
Gene map locus 18q21.3
53: LEFT-RIGHT DETERMINATION FACTOR 2; LEFTY2
LEFT-RIGHT AXIS MALFORMATIONS, INCLUDED
Gene map locus 1q42.1
54: PEROXISOME BIOGENESIS FACTOR 13; PEX13
PEROXISOME BIOGENESIS DISORDER, COMPLEMENTATION GROUP 13, INCLUDED
Gene map locus 2p15
55: GROWTH/DIFFERENTIATION FACTOR 8; GDF8
MUSCLE HYPERTROPHY, INCLUDED
Gene map locus 2q32.2
56: PEROXISOME BIOGENESIS FACTOR 12; PEX12 PEROXISOME BIOGENESIS DISORDER, COMPLEMENTATION
GROUP 3, INCLUDED; CG3, INCLUDED
Gene map locus Chr.17
57: UNCOUPLING PROTEIN 2; UCP2
OBESITY, SUSCEPTIBILITY TO, INCLUDED
Gene map locus 11q13
58: PHOSPHOLIPASE A2, GROUP VII; PLA2G7
PLATELET-ACTIVATING FACTOR ACETYLHYDROLASE DEFICIENCY, INCLUDED
Gene map locus 6p21.2-p12
59: PEROXISOME BIOGENESIS FACTOR 6; PEX6 PEROXISOME BIOGENESIS DISORDER, COMPLEMENTATION GR [symbol]
4, INCLUDED
Gene map locus 6p21.1
60: INSULIN-LIKE GROWTH FACTOR-BINDING PROTEIN, ACID-LABILE SUBUNIT;
IGFALS ACID-LABILE SUBUNIT, DEFICIENCY OF, INCLUDED
Gene map locus 16p13.3
61: POLYCYSTIC KIDNEY DISEASE 1; PKD1
POLYCYSTIN 1, INCLUDED
Gene map locus 16p13.3-p13.12
62: PATCHED, DROSOPHILA, HOMOLOG OF; PTCH
HOLOPROSENCEPHALY 7, INCLUDED; HPE7, INCLUDED
Gene map locus 9q22.3
63: CAVEOLIN 3; CAV3
MYOPATHY, DISTAL, WITH DECREASED CAVEOLIN 3, INCLUDED
Gene map locus 3p25
64: GUANIDINOACETATE METHYLTRANSFERASE; GAMT
GUANIDINOACETATE METHYLTRANSFERASE DEFICIENCY, INCLUDED
Gene map locus 19p13.3
65: CALCIUM-SENSING RECEPTOR; CASR
HYPERCALCIURIC HYPERCALCEMIA, INCLUDED
Gene map locus 3q13.3-q21
66: GLUTAMATE-CYSTEINE LIGASE, MODIFIER SUBUNIT; GCLM
MYOCARDIAL INFARCTION, SUSCEPTIBILITY TO, INCLUDED
Gene map locus 1p22.1
67: DEAFNESS, AUTOSOMAL DOMINANT NONSYNDROMIC SENSORINEURAL 5; DFNA5
DFNA5 GENE, INCLUDED; DFNA5, INCLUDED
Gene map locus 7p15
68: ARYLALKYLAMINE N-ACETYLTRANSFERASE; AANAT
DELAYED SLEEP PHASE SYNDROME, INCLUDED; DSPS, INCLUDED
Gene map locus 17q25
69: CRYSTALLIN, BETA-B1; CRYBB1
CATARACT, PULVERULENT, INCLUDED
Gene map locus 22q11.2-q12.1
70: FANCONI ANEMIA, COMPLEMENTATION GROUP E; FANCE
FANCONI ANEMIA, COMPLEMENTATION GROUP E GENE, INCLUDED
Gene map locus 6p22-p21
71: PURINERGIC RECEPTOR P2X, LIGAND-GATED ION CHANNEL, 1; P2RX1
BLEEDING DISORDER DUE TO P2RX1 DEFECT, INCLUDED
Gene map locus 17p13.3
72: MutS, E. COLI, HOMOLOG OF, 6; MSH6 COLORECTAL CANCER, HEREDITARY NONPOLYPOSIS, TYPE 5,
INCLUDED; HNPCC5, INCLUDED
Gene map locus 2p16
73: SIGNAL TRANSDUCER AND ACTIVATOR OF TRANSCRIPTION 1; STAT1
STAT1 DEFICIENCY, COMPLETE, INCLUDED
Gene map locus 2q32.2-q32.3
74: NUCLEAR RECEPTOR SUBFAMILY 4, GROUP A, MEMBER 3; NR4A3
CSMF/EWS FUSION GENE, INCLUDED
Gene map locus 9q22
75: NEUROTROPHIC TYROSINE KINASE, RECEPTOR, TYPE 2; NTRK2
OBESITY, HYPERPHAGIA, AND DEVELOPMENTAL DELAY, INCLUDED
Gene map locus 9q22.1
76: PEROXISOMAL FARNESYLATED PROTEIN; PXF PEROXISOME BIOGENESIS DISORDER, COMPLEMENTATION
GROUP 14, INCLUDED
Gene map locus 1q22
77: POSTMEIOTIC SEGREGATION INCREASED, S. CEREVISIAE, 2; PMS2 COLORECTAL CANCER, HEREDITARY
NONPOLYPOSIS, TYPE 4, INCLUDED; HNPCC4, INCLUDED
Gene map locus 7p22
78: POSTMEIOTIC SEGREGATION INCREASED, S. CEREVISIAE, 1; PMS1 COLORECTAL CANCER, HEREDITARY
NONPOLYPOSIS, TYPE 3, INCLUDED; HNPCC3, INCLUDED
Gene map locus 2q31-q33
79: BREAST CANCER 2 GENE; BRCA2
BREAST CANCER, TYPE 2, INCLUDED
Gene map locus 13q12.3
80: SOLUTE CARRIER FAMILY 1 (GLIAL HIGH AFFINITY GLUTAMATE TRANSPORTER), MEMBER 3; SLC1A3
EPISODIC ATAXIA, TYPE 6, INCLUDED; EA6, INCLUDED
Gene map locus 5p13
81. ATP-BINDING CASSETTE, SUBFAMILY A, MEMBER 1; ABCA1 CORONARY HEART DISEASE IN FAMILIAL
HYPERCHOLESTEROLEMIA, PROTECTION AGAINST, INCLUDED
Gene map locus 9q22-q31
82: ZINC FINGER PROTEIN 41; ZNF41
MENTAL RETARDATION, X-LINKED 89, INCLUDED; MRX89, INCLUDED
Gene map locus Xp22.1-cen
83: KELL BLOOD GROUP PRECURSOR; XK
MCLEOD SYNDROME, INCLUDED
Gene map locus Xp21.2-p21.1
84: XG BLOOD GROUP SYSTEM; XG
XG GLYCOPROTEIN, INCLUDED
Gene map locus Xpter-p22.32
85: THYROXINE-BINDING GLOBULIN OF SERUM; TBG
THYROXINE-BINDING GLOBULIN DEFICIENCY, INCLUDED
Gene map locus Xq22.2
86: SARCOMA, SYNOVIAL, X BREAKPOINT 1; SSX1
SARCOMA, SYNOVIAL, INCLUDED Gene map locus Xp11.2
87: RETINOSCHISIS 1, X-LINKED, JUVENILE; RS1
RETINOSCHISIS 1 GENE, INCLUDED; RS1 GENE, INCLUDED
Gene map locus Xp22.2-p22.1
88: RETINITIS PIGMENTOSA 2, X-LINKED; RP2
RP2 GENE, INCLUDED; RP2, INCLUDED
Gene map locus Xp11.3
89: PHOSPHATIDYLINOSITOL GLYCAN, CLASS A; PIGA
PAROXYSMAL NOCTURNAL HEMOGLOBINURIA, INCLUDED; PNH, INCLUDED
Gene map locus Xp22.1
90: NORRIE DISEASE; ND
NDP GENE, INCLUDED; NDP, INCLUDED
Gene map locus Xp11.4
91: MUCOPOLYSACCHARIDOSIS TYPE II
IDURONATE 2-SULFATASE, INCLUDED; IDS, INCLUDED
Gene map locus Xq28
92: MONOAMINE OXIDASE A; MAOA
BRUNNER SYNDROME, INCLUDED
Gene map locus Xp11.23
93: FRAGILE SITE MENTAL RETARDATION 1 GENE; FMR1
FRAGILE X SYNDROME, INCLUDED
Gene map locus Xq27.3
94: FRAGILE SITE, FOLIC ACID TYPE, RARE, FRA(X)(q28); FRAXE FRAGILE SITE MENTAL RETARDATION 2 GENE,
INCLUDED; FMR2, INCLUDED
Gene map locus Xq28
95: KALLMANN SYNDROME 1; KAL1 KALLMANN SYNDROME INTERVAL GENE 1, INCLUDED;
KALIG1, INCLUDED
Gene map locus Xp22.3
96: ICHTHYOSIS, X-LINKED
ARYLSULFATASE C, ISOZYME S, INCLUDED; ARSC1, INCLUDED
Gene map locus Xp22.32
97: HEMOPHILIA B; HEMB COAGULATION FACTOR IX, INCLUDED; F9, INCLUDED
Gene map locus Xq27.1-q27.2
98: HEMOPHILIA A COAGULATION FACTOR VIIIC,
PROCOAGULANT COMPONENT, INCLUDED; F8C, INCLUDED
Gene map locus Xq28
99: GONADAL DYSGENESIS, XY FEMALE TYPE; GDXY
TESTIS-DETERMINING FACTOR X-CHROMOSOMAL, INCLUDED; TDFX, INCLUDED
Gene map locus Xp22.11-p21.2
100: GLYCOGEN STORAGE DISEASE VIII
PHOSPHORYLASE KINASE, LIVER, ALPHA-2 SUBUNIT, INCLUDED; PHKA2, INCLUDED
Gene map locus Xp22.2-p22.1
101: GLUCOSE-6-PHOSPHATE DEHYDROGENASE; G6PD
ANEMIA, NONSPHEROCYTIC HEMOLYTIC, DUE TO G6PD DEFICIENCY, INCLUDED
Gene map locus Xq28
102: COLORBLINDNESS, PARTIAL, PROTAN SERIES;
CBP OPSIN1, LONG-WAVE-SENSITIVE, INCLUDED; OPN1LW, INCLUDED
Gene map locus Xq28
103: COLORBLINDNESS, PARTIAL, DEUTAN SERIES; CBD
OPSIN 1, MEDIUM-WAVE-SENSITIVE, INCLUDED; OPN1MW, INCLUDED
Gene map locus Xq28
104: FABRY DISEASE
GALACTOSIDASE, ALPHA, INCLUDED; GLA, INCLUDED
Gene map locus Xq22
105: ANEMIA, SIDEROBLASTIC, X-LINKED
DELTA-AMINOLEVULINATE SYNTHASE 2, INCLUDED; ALAS2, INCLUDED
Gene map locus Xp11.21
106: ZINC FINGER PROTEIN 674; ZNF674
MENTAL RETARDATION, X-LINKED 92, INCLUDED; MRX92, INCLUDED
Gene map locus Xp11
107: ALBINISM, OCULAR, TYPE I; OA1
OA1 GENE, INCLUDED; OA1, INCLUDED
Gene map locus Xp22.3
108: POLYGLUTAMINE-BINDING PROTEIN 1; PQBP1
GOLABI-ITO-HALL SYNDROME, INCLUDED
Gene map locus Xp11.23
109: BRUTON AGAMMAGLOBULINEMIA TYROSINE KINASE; BTK
AGAMMAGLOBULINEMIA, X-LINKED, INCLUDED; XLA, INCLUDED
Gene map locus Xq21.3-q22
110: DISCS LARGE, DROSOPHILA, HOMOLOG OF, 3; DLG3
MENTAL RETARDATION, X-LINKED 90, INCLUDED; MRX90, INCLUDED
Gene map locus Xq13.1
111: GDP DISSOCIATION INHIBITOR 1; GDI1
MENTAL RETARDATION, X-LINKED 41, INCLUDED; MRX41, INCLUDED
Gene map locus Xq28
112: ANGIOTENSIN II RECEPTOR, TYPE 2; AGTR2
MENTAL RETARDATION, X-LINKED 88, INCLUDED; MRX88, INCLUDED
Gene map locus Xq22-q23
113: FILAMIN A; FLNA
OTOPALATODIGITAL SPECTRUM DISORDER, INCLUDED
Gene map locus Xq28
114: METHYL-CpG-BINDING PROTEIN 2; MECP2
ENCEPHALOPATHY, NONPROGRESSIVE, NEONATAL-ONSET, INCLUDED
Gene map locus Xq28
115: XERODERMA PIGMENTOSUM, COMPLEMENTATION GROUP C;
XPC XPC GENE
Gene map locus 3p25
116: XERODERMA PIGMENTOSUM, COMPLEMENTATION GROUP A; XPA
XPA GENE, INCLUDED; XPA, INCLUDED
Gene map locus 9q22.3
117: WOLMAN DISEASE
LIPASE A, LYSOSOMAL ACID, INCLUDED; LIPA, INCLUDED
Gene map locus 10q24-q25
118: TYROSINEMIA, TYPE I
FUMARYLACETOACETATE HYDROLASE, INCLUDED; FAH, INCLUDED
Gene map locus 15q23-q25
119: TYROSINE TRANSAMINASE DEFICIENCY
TYROSINE AMINOTRANSFERASE, INCLUDED; TAT, INCLUDED
Gene map locus 16q22.1-q22.3
120: PROTEASE, SERINE, 1; PRSS1
TRYPSINOGEN DEFICIENCY, INCLUDED
Gene map locus 7q35
121: TREHALASE; TREH
TREHALASE DEFICIENCY, INCLUDED
122: TRANSCOBALAMIN II DEFICIENCY
TRANSCOBALAMIN II, INCLUDED; TCN2, INCLUDED
Gene map locus 22q11.2-qter
123: THYROTROPIN-RELEASING HORMONE DEFICIENCY
THYROTROPIN-RELEASING HORMONE, INCLUDED; TRH, INCLUDED
Gene map locus 3q13.3-q21
124: DIHYDROPYRIMIDINE DEHYDROGENASE; DPYD
THYMINE-URACILURIA, HEREDITARY, INCLUDED
Gene map locus 1p22
125: THROMBOXANE A SYNTHASE 1; TBXAS1
THROMBOXANE SYNTHETASE DEFICIENCY, INCLUDED
Gene map locus 7q34
126: TAY-SACHS DISEASE, AB VARIANT
GM2-ACTIVATOR, INCLUDED; GM2A, INCLUDED
Gene map locus 5q31.3-q33.1
127: PTA DEFICIENCY
COAGULATION FACTOR XI, INCLUDED; F11, INCLUDED
Gene map locus 4q35
128: PLASMIN INHIBITOR DEFICIENCY
ALPHA-2-PLASMIN INHIBITOR, INCLUDED; PLI, INCLUDED
Gene map locus 17pter-p12
129: PHOSPHOENOLPYRUVATE CARBOXYKINASE 1, SOLUBLE; PCK1
PHOSPHOENOLPYRUVATE CARBOXYKINASE DEFICIENCY, CYTOSOLIC, INCLUDED
Gene map locus 20q13.31
130: PHOSPHOGLYCERATE MUTASE, MUSCLE, DEFICIENCY OF
PHOSPHOGLYCERATE MUTASE 2, INCLUDED; PGAM2, INCLUDED
Gene map locus 7p13-p12.3
131: PHOSPHOENOLPYRUVATE CARBOXYKINASE 2, MITOCHONDRIAL; PCK2
PHOSPHOENOLPYRUVATE CARBOXYKINASE DEFICIENCY, MITOCHONDRIAL, INCLUDED
132: 6-alphaPYRUVOYLTETRAHYDROPTERIN SYNTHASE; PTS
PHENYLKETONURIA III, INCLUDED
Gene map locus 11q22.3-q23.3
133: PHENYLKETONURIA II
QUINOID DIHYDROPTERIDINE REDUCTASE, INCLUDED; QDPR, INCLUDED
Gene map locus 4p15.31
134: PHENYLKETONURIA
PHENYLALANINE HYDROXYLASE, INCLUDED; PAH, INCLUDED
Gene map locus 12q24.1
135: OSTEOPETROSIS WITH RENAL TUBULAR ACIDOSIS
CARBONIC ANHYDRASE II, INCLUDED; CA2, INCLUDED
Gene map locus 8q22
136: OROTIC ACIDURIA I
URIDINE MONOPHOSPHATE SYNTHETASE; UMPS, INCLUDED
Gene map locus 3q13
137: ORNITHINE AMINOTRANSFERASE DEFICIENCY
ORNITHINE AMINOTRANSFERASE, INCLUDED; OAT, INCLUDED
Gene map locus 10q26
138: NEURAMINIDASE DEFICIENCY WITH BETA-GALACTOSIDASE DEFICIENCY
BETA-GALACTOSIDASE PROTECTIVE PROTEIN, INCLUDED; PPGB, INCLUDED
Gene map locus 20q13.1
139: MUCOPOLYSACCHARIDOSIS TYPE VII
BETA-GLUCURONIDASE, INCLUDED; GUSB, INCLUDED
Gene map locus 7q21.11
140: MUCOPOLYSACCHARIDOSIS TYPE VI
ARYLSULFATASE B, INCLUDED; ARSB, INCLUDED
Gene map locus 5q11-q13
141: MUCOPOLYSACCHARIDOSIS TYPE IVA
GALACTOSAMINE-6-SULFATE SULFATASE, INCLUDED; GALNS, INCLUDED
Gene map locus 16q24.3
142: MEVALONATE KINASE; MVK
MEVALONIC ACIDURIA, INCLUDED
Gene map locus 12q24
143: METHIONINE ADENOSYLTRANSFERASE DEFICIENCY
METHIONINE ADENOSYLTRANSFERASE I, ALPHA, INCLUDED; MAT1A, INCLUDED
Gene map locus 10q22
144: METHEMOGLOBINEMIA DUE TO DEFICIENCY OF
METHEMOGLOBIN REDUCTASE DIAPHORASE, INCLUDED; DIA1, INCLUDED
Gene map locus 22q13.31-qter
145: METHEMOGLOBINEMIA DUE TO DEFICIENCY OF CYTOCHROME b5
CYTOCHROME b5, INCLUDED; CYB5, INCLUDED
Gene map locus 18q23
146: LIPASE, CONGENITAL ABSENCE OF PANCREATIC
PANCREATIC LIPASE, INCLUDED; PNLIP, INCLUDED
Gene map locus 10q26.1
147: LEUKOTRIENE C4 SYNTHASE; LTC4S
LEUKOTRIENE C4 SYNTHASE DEFICIENCY, INCLUDED
Gene map locus 5q35
148: 3-alphaHYDROXY-3-METHYLGLUTARYL-CoA LYASE DEFICIENCY
3-alphaHYDROXY-3-METHYLGLUTARYL-CoA LYASE, INCLUDED; HMGCL, INCLUDED
Gene map locus 1pter-p33
149: ISONIAZID INACTIVATION
ARYLAMINE N-ACETYLTRANSFERASE 2, INCLUDED; NAT2, INCLUDED
Gene map locus 8p23.1-p21.3
150: HOMOCYSTINURIA
CYSTATHIONINE BETA-SYNTHASE, INCLUDED; CBS, INCLUDED
Gene map locus 21q22.3
151: HEMOCHROMATOSIS; HFE
HFE GENE, INCLUDED; HFE, INCLUDED
Gene map locus 6p21.3
152: +234000Links
HAGEMAN FACTOR DEFICIENCY
COAGULATION FACTOR XII, INCLUDED; F12, INCLUDED
Gene map locus 5q33-qter
153: GLYCOGEN STORAGE DISEASE VII
PHOSPHOFRUCTOKINASE, MUSCLE TYPE, INCLUDED; PFKM, INCLUDED
Gene map locus 12q13.3
154: GLYCOGEN STORAGE DISEASE VI
GLYCOGEN PHOSPHORYLASE, LIVER, INCLUDED; PYGL, INCLUDED
Gene map locus 14q21-q22
155: GLYCOGEN STORAGE DISEASE III AMYLO-1,6-GLUCOSIDASE,
4-ALPHA-GLUCANOTRANSFERASE, INCLUDED; AGL, INCLUDED
Gene map locus 1p21
156: GLYCOGEN STORAGE DISEASE I
GLUCOSE-6-PHOSPHATASE, CATALYTIC, INCLUDED; G6PC, INCLUDED
Gene map locus 17q21
157: GLUTATHIONURIA
GAMMA-GLUTAMYLTRANSFERASE 1, INCLUDED; GGT1, INCLUDED
Gene map locus 22q11.1-q11.2
158: GANGLIOSIDOSIS, GENERALIZED GM1, TYPE I
GALACTOSIDASE, BETA-1, INCLUDED; GLB1, INCLUDED
Gene map locus 3p21.33
159: FUCOSIDOSIS
FUCOSIDASE, ALPHA-L, 1, INCLUDED; FUCA1, INCLUDED
Gene map locus 1p34
160: FRUCTOSURIA
KETOHEXOKINASE; KHK, INCLUDED
Gene map locus 2p23.3-p23.2
161: FRUCTOSE-1,6-BISPHOSPHATASE 1; FBP1
FRUCTOSE-1,6-BISPHOSPHATASE DEFICIENCY, INCLUDED
Gene map locus 9q22.2-q22.3
162: FRUCTOSE INTOLERANCE, HEREDITARY
ALDOLASE B, FRUCTOSE-BISPHOSPHATE, INCLUDED; ALDOB, INCLUDED
Gene map locus 9q22.3
163: PREKALLIKREIN DEFICIENCY
KALLIKREIN B, PLASMA, INCLUDED; KLKB1, INCLUDED
Gene map locus 4q35
164: FLAUJEAC FACTOR DEFICIENCY
KININOGEN DEFICIENCY, TOTAL, INCLUDED
Gene map locus 3q27
165: FARBER LIPOGRANULOMATOSIS
N-ACYLSPHINGOSINE AMIDOHYDROLASE, INCLUDED; ASAH, INCLUDED
Gene map locus 8p22-p21.3
166: FANCONI ANEMIA, COMPLEMENTATION GROUP D2; FANCD2
FANCD2 GENE
Gene map locus 3p25.3
167: FANCONI ANEMIA, COMPLEMENTATION GROUP C; FANCC
FANCC GENE, INCLUDED
Gene map locus 9q22.3
168: FACTOR X DEFICIENCY
COAGULATION FACTOR X, INCLUDED; F10, INCLUDED
Gene map locus 13q34
169: FACTOR VII DEFICIENCY
COAGULATION FACTOR VII, INCLUDED; F7, INCLUDED
Gene map locus 13q34
170: FACTOR V DEFICIENCY
COAGULATION FACTOR V, INCLUDED; F5, INCLUDED
Gene map locus 1q23
171: DIPHOSPHOGLYCERATE MUTASE DEFICIENCY OF ERYTHROCYTE
2,3-alphaBISPHOSPHOGLYCERATE PHOSPHATASE; BPGM, INCLUDED
Gene map locus 7q31-q34
172: DIHYDROPYRIMIDINASE; DPYS
DIHYDROPYRIMIDINURIA, INCLUDED
Gene map locus 8q22
173: 2,4-alphaDIENOYL-CoA REDUCTASE 1; DECR1
2,4-alphaDIENOYL-CoA REDUCTASE DEFICIENCY, INCLUDED
Gene map locus 8q21.3
174: DIABETES MELLITUS, INSULIN-DEPENDENT; IDDM
DIABETES MELLITUS, INSULIN-DEPENDENT, 1, INCLUDED; IDDM1, INCLUDED
Gene map locus Xp11.23-q13.3, 12q24.2, 12q24.2, 1p13, 6p21.3
175: MITOCHONDRIAL DEAFNESS MODIFIER GENE 1; MDM1
DEAFNESS, SENSORINEURAL, AUTOSOMAL-MITOCHONDRIAL TYPE, INCLUDED
Gene map locus Chr.8
176: CORTISOL 11-BETA-KETOREDUCTASE DEFICIENCY
11-alphaBETA-HYDROXYSTEROID DEHYDROGENASE, TYPE II, INCLUDED; HSD11B2, INCLUDED
Gene map locus 16q22
177: COMPLEMENT COMPONENT 6 DEFICIENCY
COMPLEMENT COMPONENT 6, INCLUDED; C6, INCLUDED
Gene map locus 5p13
178: I FACTOR; IF
COMPLEMENT COMPONENT 3 INACTIVATOR, DEFICIENCY OF, INCLUDED
Gene map locus 4q25
179: COMPLEMENT COMPONENT 2 DEFICIENCY
COMPLEMENT COMPONENT 2, INCLUDED; C2, INCLUDED
Gene map locus 6p21.3
180: COMPLEMENT COMPONENT C1r DEFICIENCY
COMPLEMENT COMPONENT 1, r SUBCOMPONENT, INCLUDED; C1R, INCLUDED
Gene map locus 12p13
181: SOLUTE CARRIER FAMILY 25 (CARNITINE/ACYLCARNITINE TRANSLOCASE),
MEMBER 20; SLC25A20 CARNITINE-ACYLCARNITINE TRANSLOCASE DEFICIENCY, INCLUDED
Gene map locus 3p21.31
182: FUCOSYLTRANSFERASE 1; FUT1
BOMBAY PHENOTYPE, INCLUDED
Gene map locus 19q13.3
183: ASPARTYLGLUCOSAMINURIA
ASPARTYLGLUCOSAMINIDASE, INCLUDED; AGA, INCLUDED
Gene map locus 4q32-q33
184: ALPHA-KETOGLUTARATE DEHYDROGENASE DEFICIENCY
OXOGLUTARATE DEHYDROGENASE, INCLUDED; OGDH, INCLUDED
Gene map locus 7p14-p13
185: OCULOCUTANEOUS ALBINISM, TYPE II; OCA2
OCA2 GENE, INCLUDED
Gene map locus 15q11.2-q12
186: ADRENAL HYPERPLASIA, CONGENITAL, DUE TO 11-BETA-HYDROXYLASE DEFICIENCY
CYTOCHROME P450, SUBFAMILY XIB, POLYPEPTIDE 1, INCLUDED; CYP11B1, INCLUDED
Gene map locus 8q21
187: ADRENAL HYPERPLASIA, CONGENITAL, DUE TO 21-HYDROXYLASE DEFICIENCY
CYTOCHROME P450, SUBFAMILY XXIA, POLYPEPTIDE 2, INCLUDED; CYP21A2, INCLUDED
Gene map locus 6p21.3
188: ADRENAL HYPERPLASIA II
3-alphaBETA-HYDROXYSTEROID DEHYDROGENASE/DELTA-ISOMERASE, TYPE II, INCLUDED
Gene map locus 1p13.1
189: ACETYL-CoA CARBOXYLASE-ALPHA; ACACA
ACETYL-CoA CARBOXYLASE DEFICIENCY, INCLUDED
Gene map locus 17q21
190: VON WILLEBRAND DISEASE
VON WILLEBRAND FACTOR, INCLUDED; VWF, INCLUDED
Gene map locus 12p13.3
191: R BINDER PROTEIN
R BINDER DEFICIENCY, INCLUDED
192: INTEGRIN, ALPHA-2; ITGA2
PLATELET RECEPTOR FOR COLLAGEN, DEFICIENCY OF, INCLUDED
Gene map locus 5q23-q31
193: UBIQUITIN CARBOXYL-TERMINAL ESTERASE L1; UCHL1
PARKINSON DISEASE 5, INCLUDED; PARK5, INCLUDED
Gene map locus 4p14
194: TUMOR PROTEIN p53; TP53
COLON TUMORS, CONCURRENT MULTIPLE PRIMARY, INCLUDED
Gene map locus 17p13.1
195: TROPONIN I, CARDIAC; TNNI3
CARDIOMYOPATHY, FAMILIAL HYPERTROPHIC, 7, INCLUDED; CMH7, INCLUDED
Gene map locus 19q13.4
196: TRITANOPIA
OPSIN 1, SHORT-WAVE-SENSITIVE, INCLUDED; OPN1SW, INCLUDED
Gene map locus 7q31.3-q32
197: TRIOSEPHOSPHATE ISOMERASE 1; TPI1
TRIOSEPHOSPHATE ISOMERASE DEFICIENCY, INCLUDED
Gene map locus 12p13
198: TRANSFORMING GROWTH FACTOR-BETA RECEPTOR, TYPE II; TGFBR2
COLORECTAL CANCER, HEREDITARY NONPOLYPOSIS, TYPE 6, INCLUDED; HNPCC6, INCLUDED
Gene map locus 3p22
199: TITIN; TTN
CARDIOMYOPATHY, FAMILIAL HYPERTROPHIC, 9, INCLUDED; CMH9, INCLUDED
Gene map locus 2q24.3
200: THYROTROPIN-RELEASING HORMONE RECEPTOR; TRHR
THYROTROPIN-RELEASING HORMONE RESISTANCE, GENERALIZED, INCLUDED
Gene map locus 8q23
201: THYROGLOBULIN; TG
GOITER, FAMILIAL, WITH HYPOTHYROIDISM, AUTOSOMAL RECESSIVE, INCLUDED
Gene map locus 8q24.2-q24.3
202: THROMBOXANE A2 RECEPTOR, PLATELET; TBXA2R
BLEEDING DISORDER DUE TO DEFECTIVE THROMBOXANE A2 RECEPTOR, INCLUDED
Gene map locus 19p13.3
203: THROMBOMODULIN; THBD
THROMBOPHILIA DUE TO THROMBOMODULIN DEFECT, INCLUDED
Gene map locus 20p11.2
204: TERATOCARCINOMA-DERIVED GROWTH FACTOR 1; TDGF1
FOREBRAIN DEFECTS, INCLUDED
Gene map locus 3p23-p21
205: CD4 ANTIGEN; CD4
CD4+ LYMPHOCYTE DEFICIENCY, INCLUDED
Gene map locus 12pter-p12
206: LIM DOMAIN ONLY 1; LMO1
T-CELL ACUTE LYMPHOBLASTIC LEUKEMIA, INCLUDED
Gene map locus 11p15
207: CD3 ANTIGEN, EPSILON SUBUNIT; CD3E
IMMUNODEFICIENCY DUE TO DEFECT IN CD3-EPSILON, INCLUDED
Gene map locus 11q23
208: T-CELL LEUKEMIA, HOMEOBOX 1; TLX1
T-CELL ACUTE LYMPHOCYTIC LEUKEMIA, INCLUDED
Gene map locus 10q24
209: CD3 ANTIGEN, GAMMA SUBUNIT; CD3G
IMMUNODEFICIENCY DUE TO DEFECT IN CD3-GAMMA, INCLUDED
Gene map locus 11q23
210: MATRIX METALLOPROTEINASE 3; MMP3
CORONARY HEART DISEASE, SUSCEPTIBILITY TO, INCLUDED
Gene map locus 11q23
211: NUCLEAR RECEPTOR SUBFAMILY 5, GROUP A, MEMBER 1; NR5A1
SEX REVERSAL, XY, WITH ADRENAL FAILURE, INCLUDED
Gene map locus 9q33
212: SPHEROCYTOSIS, HEREDITARY; HS
ANKYRIN 1, INCLUDED; ANK1, INCLUDED
Gene map locus 8p11.2
213: SPECTRIN, BETA, ERYTHROCYTIC; SPTB
SPHEROCYTOSIS, TYPE I, INCLUDED; SPH1, INCLUDED
Gene map locus 14q22-q23.2
214: SOLUTE CARRIER FAMILY 34 (SODIUM/PHOSPHATE COTRANSPORTER), MEMBER 1; SLC34A1 UROLITHIASI [symbol]
HYPOPHOSPATEMIC, INCLUDED
Gene map locus 5q35
215: SEPIAPTERIN REDUCTASE; SPR
Gene map locus 2p14-p12
216: FUCOSYLTRANSFERASE 2; FUT2
NORWALK VIRUS INFECTION, RESISTANCE TO, INCLUDED
Gene map locus 19q13.3
217: S-ADENOSYLHOMOCYSTEINE HYDROLASE; AHCY HYPERMETHIONINEMIA WITH DEFICIENCY OF S-
ADENOSYLHOMOCYSTEINE HYDROLASE, INCLUDED
Gene map locus 20cen-q13.1
218: POLYMERASE II, RNA, SUBUNIT A; POLR2A
ALPHA-AMANITIN RESISTANCE, INCLUDED
Gene map locus 17p13.1
219: RHODOPSIN; RHO
RETINITIS PIGMENTOSA 4, INCLUDED; RP4, INCLUDED
Gene map locus 3q21-q24
220: RETINOL-BINDING PROTEIN 4; RBP4
RETINOL-BINDING PROTEIN DEFICIENCY, INCLUDED
Gene map locus 10q24
221: RETINOBLASTOMA; RB1
OSTEOSARCOMA, RETINOBLASTOMA-RELATED, INCLUDED
Gene map locus 13q14.1-q14.2
222: RETINAL PIGMENT EPITHELIUM-SPECIFIC PROTEIN, 65-KD; RPE65
RETINITIS PIGMENTOSA 20, INCLUDED; RP20, INCLUDED
Gene map locus 1p31
223: PYRUVATE DEHYDROGENASE, BETA POLYPEPTIDE; PDHB
PYRUVATE DEHYDROGENASE E1-BETA DEFICIENCY, INCLUDED
Gene map locus 3p13-q23
224: BUTYRYLCHOLINESTERASE; BCHE
SUXAMETHONIUM SENSITIVITY, INCLUDED
Gene map locus 3q26.1-q26.2
225: PROTOPORPHYRIA, ERYTHROPOIETIC
FERROCHELATASE, INCLUDED; FECH, INCLUDED
Gene map locus 18q21.3
226: ZETA-CHAIN-ASSOCIATED PROTEIN KINASE; ZAP70
SELECTIVE T-CELL DEFECT, INCLUDED; STD, INCLUDED
Gene map locus 2q12
227: COAGULATION FACTOR II; F2
HYPOPROTHROMBINEMIA, INCLUDED
Gene map locus 11p11-q12
228: PROTEIN S, ALPHA; PROS1
PROTEIN S ALPHA DEFICIENCY, INCLUDED
Gene map locus 3p11.1-q11.2
229: PROTEIN C DEFICIENCY, CONGENITAL THROMBOTIC DISEASE DUE TO
PROTEIN C, INCLUDED; PROC, INCLUDED
Gene map locus 2q13-q14
230: PROSAPOSIN; PSAP
SAPOSIN A, INCLUDED
Gene map locus 10q22.1
231: INSULIN; INS
HYPERPROINSULINEMIA, INCLUDED
Gene map locus 11p15.5
232: TRANSTHYRETIN; TTR
AMYLOIDOSIS I, INCLUDED
Gene map locus 18q11.2-q12.1
233: POTASSIUM CHANNEL, VOLTAGE-GATED, ISK-RELATED SUBFAMILY, MEMBER 1;
KCNE1 LONG QT SYNDROME 5, INCLUDED; LQT5, INCLUDED
Gene map locus 21q22.1-q22.2
234: PORPHYRIA CUTANEA TARDA
PORPHYRIA, HEPATOERYTHROPOIETIC, INCLUDED; HEP, INCLUDED
Gene map locus 1p34
235: ADENOMATOUS POLYPOSIS OF THE COLON; APC
GARDNER SYNDROME, INCLUDED; GS, INCLUDED
Gene map locus 5q21-q22
236: POLYCYSTIC KIDNEY DISEASE 2; PKD2
POLYCYSTIN 2, INCLUDED
Gene map locus 4q21-q23
237: POLIOVIRUS RECEPTOR; PVR
POLIOVIRUS, SUSCEPTIBILITY TO, INCLUDED
Gene map locus 19q13.2-q13.3
238: SELECTIN P; SELP
PLATELET ALPHA/DELTA STORAGE POOL DEFICIENCY, INCLUDED
Gene map locus 1q23-q25
239: INTEGRIN, BETA-3; ITGB3
PLATELET-SPECIFIC ANTIGEN SYSTEM PL(A1), INCLUDED
Gene map locus 17q21.32
240: PLASMINOGEN ACTIVATOR, TISSUE; PLAT
PLASMINOGEN ACTIVATOR, TISSUE TYPE, INCREASE IN, INCLUDED
Gene map locus 8p12
241: PLASMINOGEN; PLG
PLASMINOGEN DEFICIENCY, INCLUDED
Gene map locus 6q26
242: POU DOMAIN, CLASS 1, TRANSCRIPTION FACTOR 1; POU1F1
PITUITARY HORMONE DEFICIENCY, COMBINED, INCLUDED; CPHD, INCLUDED
Gene map locus 3p11
243: PHOSPHOSERINE PHOSPHATASE; PSPH
PHOSPHOSERINE PHOSPHATASE DEFICIENCY, INCLUDED
Gene map locus 7p15.2-p15.1
244: PHOSPHORYLASE KINASE, TESTIS/LIVER, GAMMA-2; PHKG2
CIRRHOSIS DUE TO LIVER PHOSPHORYLASE KINASE DEFICIENCY, INCLUDED
Gene map locus 16p12.1-p11.2
245: METHYLENETETRAHYDROFOLATE DEHYDROGENASE 1; MTHFD1
ABRUPTIO PLACENTAE, SUSCEPTIBILITY TO, INCLUDED
Gene map locus 14q24
246: PEROXISOME PROLIFERATOR-ACTIVATED RECEPTOR-ALPHA; PPARA
HYPERAPOBETALIPOPROTEINEMIA, SUSCEPTIBILITY TO, INCLUDED
Gene map locus 22q12-q13.1
247: ATP-BINDING CASSETTE, SUBFAMILY D, MEMBER 3; ABCD3
ZELLWEGER SYNDROME 2, INCLUDED; ZWS2, INCLUDED
Gene map locus 1p22-p21
248: PEROXISOMAL MEMBRANE PROTEN 3; PXMP3
ZELLWEGER SYNDROME 3, INCLUDED; ZWS3, INCLUDED
Gene map locus 8q21.1
249: PEPTIDASE D; PEPD
PROLIDASE DEFICIENCY, INCLUDED
Gene map locus 19cen-q13.11
250: PARAOXONASE 1; PON1
ORGANOPHOSPHATE POISONING, SENSITIVITY TO, INCLUDED
Gene map locus 7q21.3
251: PARATHYROID HORMONE-LIKE HORMONE; PTHLH
HUMORAL HYPERCALCEMIA OF MALIGNANCY, INCLUDED; HHM, INCLUDED
Gene map locus 12p12.1-p11.2
252: GLI-KRUPPEL FAMILY MEMBER 2; GLI2
PITUITARY ANOMALIES WITH HOLOPROSENCEPHALY-LIKE FEATURES, INCLUDED
Gene map locus 2q14
253: V-RAF-1 MURINE LEUKEMIA VIRAL ONCOGENE HOMOLOG 1; RAF1
PAROTID GLAND TUMORS, INCLUDED
Gene map locus 3p25
254: NUCLEOSIDE PHOSPHORYLASE; NP
NUCLEOSIDE PHOSPHORYLASE DEFICIENCY, INCLUDED
Gene map locus 14q13.1
255: NUCLEAR FACTOR OF KAPPA LIGHT CHAIN GENE ENHANCER IN B CELLS INHIBITOR, ALPHA; NFKBIA
ECTODERMAL DYSPLASIA, ANHIDROTIC, WITH T-CELL IMMUNODEFICIENCY,
AUTOSOMAL DOMINANT, INCLUDED
Gene map locus 14q13
256: NITRIC OXIDE SYNTHASE 3; NOS3
CORONARY SPASMS, SUSCEPTIBILITY TO, INCLUDED
Gene map locus 7q36
257: NEUROFIBROMATOSIS, TYPE I; NF1
NEUROFIBROMIN, INCLUDED
Gene map locus 17q11.2, 2p22-p21
258: NEURAL RETINA LEUCINE ZIPPER; NRL
RETINITIS PIGMENTOSA 27, INCLUDED; RP27, INCLUDED
Gene map locus 14q11.1-q11.2
259: MYOSIN, HEAVY CHAIN 6, CARDIAC MUSCLE, ALPHA; MYH6
ATRIAL SEPTAL DEFECT 3, INCLUDED; ASD3, INCLUDED
Gene map locus 14q12
260: MYELOID/LYMPHOID OR MIXED LINEAGE LEUKEMIA; MLL
MLL/GMPS FUSION GENE, INCLUDED
Gene map locus 11q23
281: INSULIN-LIKE GROWTH FACTOR II; IGF2
IGF-II OVERGROWTH SYNDROME, INCLUDED
Gene map locus 11p15.5
282: IMMUNOGLOBULIN KAPPA CONSTANT REGION; IGKC
KAPPA-CHAIN DEFICIENCY, INCLUDED
Gene map locus 2p12
283: HUNTINGTON DISEASE; HD
HUNTINGTIN, INCLUDED; HD, INCLUDED
Gene map locus 4p16.3
284: HOMEOBOX D10; HOXD10
CHARCOT-MARIE-TOOTH DISEASE, FOOT DEFORMITY OF, INCLUDED
Gene map locus 2q31-q32
285: HOMEOBOX D4; HOXD4
LEUKEMIA, ACUTE LYMPHOBLASTIC, SUSCEPTIBILITY TO, INCLUDED
Gene map locus 2q31-q32
286: 3-alphaHYDROXY-3-METHYLGLUTARYL-CoA REDUCTASE; HMGCR
STATINS, ATTENUATED CHOLESTEROL LOWERING BY, INCLUDED
Gene map locus 5q13.3-q14
287: HISTIDINE-RICH GLYCOPROTEIN; HRG
THROMBOPHILIA DUE TO ELEVATED HRG, INCLUDED
Gene map locus 3q27
288: HEPARIN COFACTOR II; HCF2
HCF II DEFICIENCY, INCLUDED
Gene map locus 22q11
289: HEMOGLOBIN--BETA LOCUS; HBB
BETA-THALASSEMIAS, INCLUDED
Gene map locus 11p15.5
290: HEMOGLOBIN--ALPHA LOCUS 1; HBA1
ALPHA-THALASSEMIAS, INCLUDED
Gene map locus 16pter-p13.3
291: HEME OXYGENASE 1; HMOX1
HEME OXYGENASE 1 DEFICIENCY, INCLUDED
Gene map locus 22q12
292: GUANINE NUCLEOTIDE-BINDING PROTEIN, ALPHA-TRANSDUCING ACTIVITY
POLYPEPTIDE 2; GNAT2 ACHROMATOPSIA 4, INCLUDED; ACHM4, INCLUDED
Gene map locus 1p13
293: GNAS COMPLEX LOCUS; GNAS
PROLONGED BLEEDING TIME, BRACHYDACTYLY, AND MENTAL RETARDATION, INCLUDED
Gene map locus 20q13.2
294: GROWTH HORMONE 1; GH1
GROWTH HORMONE DEFICIENCY, INCLUDED; GHD, INCLUDED
Gene map locus 17q22-q24
295: GROUP-SPECIFIC COMPONENT; GC
GRAVES DISEASE, SUSCEPTIBILITY TO, 3, INCLUDED
Gene map locus 4q12
296: HYDROXYACYL GLUTATHIONE HYDROLASE; HAGH
GLYOXALASE II DEFICIENCY, INCLUDED
Gene map locus 16p13
297: GLUTATHIONE PEROXIDASE; GPX1
GLUTATHIONE PEROXIDASE DEFICIENCY, HEMOLYTIC ANEMIA POSSIBLY DUE TO, INCLUDED
Gene map locus 3p21.3
298: GLUTATHIONE REDUCTASE; GSR GLUTATHIONE REDUCTASE, HEMOLYTIC ANEMIA DUE TO DEFICIENCY [symbol]
IN RED CELLS, INCLUDED
Gene map locus 8p21.1
299: 1-alphaPYRROLINE-5-CARBOXYLATE SYNTHETASE; PYCS HYPERAMMONEMIA WITH HYPOORNITHINEMIA,
HYPOCITRULLINEMIA, HYPOARGININEMIA, AND HYPOPROLINEMIA, INCLUDED
Gene map locus 10q24.3
300: GLUCOCORTICOID RECEPTOR; GCCR
GLUCOCORTICOID RECEPTOR DEFICIENCY, INCLUDED
Gene map locus 5q31
301: 4-αAMINOBUTYRATE AMINOTRANSFERASE; ABAT
GABA-TRANSAMINASE DEFICIENCY, INCLUDED
Gene map locus 16p13.3
302: FUCOSYLTRANSFERASE 6; FUT6
Gene map locus 19p13.3
303: FUCOSIDASE, ALPHA-L, 2; FUCA2
FUCOSIDASE, PLASMA, LOW, INCLUDED
Gene map locus 6q25-qter
304: FOLYLPOLYGLUTAMATE SYNTHETASE; FPGS
GAT-MINUS AUXOTROPH, INCLUDED
Gene map locus 9cen-q34
305: FIBRINOGEN, A ALPHA POLYPEPTIDE; FGA
DYSFIBRINOGENEMIA CAUSING RECURRENT THROMBOSIS, INCLUDED
Gene map locus 4q28
306: FERRITIN HEAVY CHAIN 1; FTH1
IRON OVERLOAD, AUTOSOMAL DOMINANT, INCLUDED
Gene map locus 11q12-q13
307: FACTOR XIII, B SUBUNIT; F13B
FACTOR XIII, B SUBUNIT, DEFICIENCY OF, INCLUDED
Gene map locus 1q31-q32.1
308: FACTOR XIII, A1 SUBUNIT; F13A1
FACTOR XIII, A SUBUNIT, DEFICIENCY OF, INCLUDED
Gene map locus 6p25-p24
309: EXCISION-REPAIR, COMPLEMENTING DEFECTIVE, IN CHINESE HAMSTER, 5; ERCC5
XERODERMA PIGMENTOSUM/COCKAYNE SYNDROME COMPLEX, INCLUDED
Gene map locus 13q33
310: EXCISION-REPAIR, COMPLEMENTING DEFECTIVE, IN CHINESE HAMSTER, 3; ERCC3
XERODERMA PIGMENTOSUM II, INCLUDED; XP2, INCLUDED
Gene map locus 2q21
311: EWING SARCOMA BREAKPOINT REGION 1; EWSR1
EWING SARCOMA, INCLUDED; EWS, INCLUDED; ES, INCLUDED
Gene map locus 22q12
312: EPOXIDE HYDROLASE 1, MICROSOMAL; EPHX1
PHENYTOIN TOXICITY, INCLUDED
Gene map locus 1q42.1
313: ENOLASE 3; ENO3
ENOLASE 3 DEFICIENCY, INCLUDED
Gene map locus 17pter-p12
314: MULTIPLE ENDOCRINE NEOPLASIA, TYPE I; MEN1
MENIN, INCLUDED
Gene map locus 11q13
315: ERYTHROCYTE MEMBRANE PROTEIN BAND 4.1; EPB41
ELLIPTOCYTOSIS 1, INCLUDED; EL1, INCLUDED
Gene map locus 1p36.2-p34
316: DOPAMINE RECEPTOR D5; DRD5
DYSTONIA, PRIMARY CERVICAL, INCLUDED
Gene map locus 4p16.1-p15.3
317: DNA DAMAGE-INDUCIBLE TRANSCRIPT 3; DDIT3
MYXOID LIPOSARCOMA, INCLUDED
Gene map locus 12q13.1-q13.2
318: DIHYDROFOLATE REDUCTASE; DHFR
DIHYDROFOLATE REDUCTASE PSEUDOGENES, INCLUDED
Gene map locus 5q11.2-q13.2
319: DESMOPLAKIN; DSP
DESMOPLAKIN I, INCLUDED; DP1, INCLUDED
Gene map locus 6p24
320: DELTA-AMINOLEVULINATE DEHYDRATASE; ALAD
DELTA-AMINOLEVULINATE DEHYDRATASE DEFICIENCY, INCLUDED
Gene map locus 9q34
321: DECAY-ACCELERATING FACTOR FOR COMPLEMENT; DAF
CROMER BLOOD GROUP, INCLUDED; CROM, INCLUDED
Gene map locus 1q32
322: CYTOCHROME P450, SUBFAMILY XIB, POLYPEPTIDE 2; CYP11B2 HYPERRENINEMIC HYPOALDOSTERONIS [symbol]
FAMILIAL, 1, INCLUDED; FHHA1, INCLUDED
Gene map locus 8q21
323: CYTOCHROME P450, SUBFAMILY I, POLYPEPTIDE 2; CYP1A2
PHENACETIN METABOLISM, DEFECT IN, INCLUDED
Gene map locus 15q22-qter
324: CYTOCHROME P450, SUBFAMILY IID, POLYPEPTIDE 6;
CYP2D6
Gene map locus 22q13.1
325: CRYSTALLIN, GAMMA-D; CRYGD
CATARACT, PUNCTATE, PROGRESSIVE JUVENILE-ONSET, INCLUDED
Gene map locus 2q33-q35
326: CRYSTALLIN, GAMMA-C; CRYGC
CATARACT, VARIABLE ZONULAR PULVERULENT, INCLUDED
Gene map locus 2q33-q35
327: CRYSTALLIN, ALPHA-B; CRYAB
CATARACT, POSTERIOR POLAR 2, INCLUDED
Gene map locus 11q22.3-q23.1
328: CRYSTALLIN, ALPHA-A; CRYAA
CATARACT, ZONULAR CENTRAL NUCLEAR, INCLUDED
Gene map locus 21q22.3
329: CYTOCHROME P450, SUBFAMILY IIA, POLYPEPTIDE 6; CYP2A6
TEGAFUR, POOR METABOLISM OF, INCLUDED
Gene map locus 19q13.2
330: CORTICOTROPIN-RELEASING HORMONE; CRH
CORTICOTROPIN-RELEASING HORMONE DEFICIENCY, INCLUDED
Gene map locus 8q13
331: CORTICOSTEROID-BINDING GLOBULIN; CBG
CORTICOSTEROID-BINDING GLOBULIN DEFICIENCY, INCLUDED
Gene map locus 14q32.1
332: COPROPORPHYRIA
COPROPORPHYRINOGEN OXIDASE, INCLUDED; CPO, INCLUDED
Gene map locus 3q12
333: CONTRACTURAL ARACHNODACTYLY, CONGENITAL
FIBRILLIN 2, INCLUDED; FBN2, INCLUDED
Gene map locus 5q23-q31
334: COMPLEMENT COMPONENT 8 DEFICIENCY, TYPE II
COMPLEMENT COMPONENT 8, BETA SUBUNIT, INCLUDED; C8B, INCLUDED
Gene map locus 1p32
335: COMPLEMENT COMPONENT 8 DEFICIENCY, TYPE I
COMPLEMENT COMPONENT 8, ALPHA SUBUNIT, INCLUDED; C8A, INCLUDED
Gene map locus 1p32
336: COMPLEMENT COMPONENT 9; C9
COMPLEMENT COMPONENT 9 DEFICIENCY, INCLUDED
Gene map locus 5p13
337: MEMBRANE COFACTOR PROTEIN; MCP
MEASLES, SUSCEPTIBILITY TO, INCLUDED
Gene map locus 1q32
338: COMPLEMENT COMPONENT 5; C5
LIVER FIBROSIS, INCLUDED
Gene map locus 9q34.1
339: COMPLEMENT COMPONENT 4A; C4A
COMPLEMENT COMPONENT 4A DEFICIENCY, INCLUDED
Gene map locus 6p21.3
340: COMPLEMENT COMPONENT 3; C3
C3b, INCLUDED
Gene map locus 19p13.3-p13.2
341: COMPLEMENT COMPONENT 1, s SUBCOMPONENT; C1S
COMPLEMENT COMPONENT C1s DEFICIENCY, INCLUDED
Gene map locus 12p13
342: COMPLEMENT COMPONENT 1, q SUBCOMPONENT, BETA POLYPEPTIDE; C1QB
C1q DEFICIENCY, INCLUDED
Gene map locus 1p36.3-p34.1
343: COLLAGEN, TYPE IX, ALPHA-1; COL9A1
EPIPHYSEAL DYSPLASIA, MULTIPLE, COL9A1-RELATED
Gene map locus 6q13
344: COLLAGEN, TYPE II, ALPHA-1; COL2A1
CHONDROCALCIN, INCLUDED
Gene map locus 12q13.11-q13.2
345: CHOLINERGIC RECEPTOR, NEURONAL NICOTINIC, ALPHA POLYPEPTIDE 7; CHRNA7
SCHIZOPHRENIA, NEUROPHYSIOLOGIC DEFECT IN, INCLUDED
Gene map locus 15q14
346: CATECHOL-O-METHYLTRANSFERASE; COMT
CATECHOL-O-METHYLTRANSFERASE ACTIVITY, LOW, IN RED CELLS, INCLUDED
Gene map locus 22q11.2
347: CATALASE; CAT
ACATALASEMIA, INCLUDED
Gene map locus 11p13
348: CARBOXYLESTERASE 1; CES1
MONOCYTE ESTERASE DEFICIENCY, INCLUDED
Gene map locus 16q13-q22.1
349: COLLAGEN, TYPE XVII, ALPHA-1; COL17A1
EPIDERMOLYSIS BULLOSA, JUNCTIONAL, LOCALISATA VARIANT, INCLUDED
Gene map locus 10q24.3
350: BREAST CANCER 1 GENE; BRCA1
BREAST CANCER, TYPE 1, INCLUDED
Gene map locus 17q21
351: BLOOD GROUP--Ss LOCUS; Ss
GLYCOPHORIN B, INCLUDED; GPB, INCLUDED
Gene map locus 4q28-q31
352: BETA-1,4-N-ACETYL-GALACTOSAMINYLTRANSFERASE 2; B4GALNT2
BLOOD GROUP--Sd SYSTEM, INCLUDED; SD, INCLUDED
353: RHESUS BLOOD GROUP, CcEe ANTIGENS; RHCE
RH-NULL DISEASE, AMORPH TYPE, INCLUDED
Gene map locus 1p36.2-p34
354: BLOOD GROUP--MN LOCUS; MN
GLYCOPHORIN A, INCLUDED; GPA, INCLUDED
Gene map locus 4q28.2-q31.1
355: LANDSTEINER-WIENER BLOOD GROUP; LW
Gene map locus 19p13.3
356: BLOOD GROUP--LUTHERAN SYSTEM; LU
AUBERGER SYSTEM, INCLUDED; AU, INCLUDED
Gene map locus 19q13.2
357: FUCOSYLTRANSFERASE 3; FUT3
BLOOD GROUP--LEWIS SYSTEM; Le
Gene map locus 19p13.3
358: BLOOD GROUP--KELL-CELLANO SYSTEM; KEL
KELL NULL, INCLUDED; Ko, INCLUDED
Gene map locus 7q33
359: BLOOD GROUP--GERBICH; Ge
GLYCOPHORIN C, INCLUDED; GYPC, INCLUDED; GPC, INCLUDED
Gene map locus 2q14-q21
360: BLOOD GROUP--DUFFY SYSTEM; FY
GLYCOPROTEIN D, INCLUDED; GPD, INCLUDED
Gene map locus 1q21-q22
361: ADP-RIBOSYLTRANSFERASE 4; ART4
BLOOD GROUP--DOMBROCK SYSTEM, INCLUDED; DO, INCLUDED
Gene map locus 12p13-p12
362: ABO BLOOD GROUP; ABO
ABO HISTO-BLOOD GROUP GLYCOSYLTRANSFERASES, INCLUDED
Gene map locus 9q34
363: BETA-2-ADRENERGIC RECEPTOR; ADRB2
BETA-2-ADRENORECEPTOR AGONIST, REDUCED RESPONSE TO, INCLUDED
Gene map locus 5q32-q34
364: SOLUTE CARRIER FAMILY 4 (ANION EXCHANGER), MEMBER 1; SLC4A1
ACANTHOCYTOSIS, ONE FORM OF, INCLUDED
Gene map locus 17q21-q22
365: CYTOCHROME P450, FAMILY 19, SUBFAMILY A, POLYPEPTIDE 1; CYP19A1
AROMATASE DEFICIENCY, INCLUDED
Gene map locus 15q21.1
366: APOLIPOPROTEIN E; APOE
APOLIPOPROTEIN E, DEFICIENCY OR DEFECT OF, INCLUDED
Gene map locus 19q13.2
367: APOLIPOPROTEIN B; APOB
APOB100, INCLUDED
Gene map locus 2p24
368: PROTEASE INHIBITOR 1; PI
ALPHA-1-ANTITRYPSIN DEFICIENCY, AUTOSOMAL
RECESSIVE, INCLUDED
Gene map locus 14q32.1
369: ANTITHROMBIN III DEFICIENCY
ANTITHROMBIN III, INCLUDED; AT3, INCLUDED
Gene map locus 1q23-q25
370: CD59 ANTIGEN P18-20; CD59
CD59 DEFICIENCY, INCLUDED
Gene map locus 11p13
371: CD19 ANTIGEN; CD19
ANTIBODY DEFICIENCY DUE TO DEFECT IN CD19, INCLUDED
Gene map locus 16p11.2
372: ANGIOTENSIN I-CONVERTING ENZYME; ACE
ANGIOTENSIN I-CONVERTING ENZYME, TESTICULAR, INCLUDED
Gene map locus 17q23
373: ALPHA-FETOPROTEIN; AFP
ALPHA-FETOPROTEIN, HEREDITARY PERSISTENCE OF, INCLUDED; HPAFP, INCLUDED
Gene map locus 4q11-q13
374: ALPHA-2-MACROGLOBULIN; A2M
ALPHA-2-MACROGLOBULIN DEFICIENCY, INCLUDED
Gene map locus 12p13.3-p12.3
375: ALDOLASE A, FRUCTOSE-BISPHOSPHATE; ALDOA
ALDOLASE A DEFICIENCY, INCLUDED
Gene map locus 16q22-q24
376: ALBUMIN; ALB
DYSALBUMINEMIC HYPERTHYROXINEMIA, INCLUDED
Gene map locus 4q11-q13
377: ADENYLATE KINASE 1; AK1
ADENYLATE KINASE DEFICIENCY, INCLUDED
Gene map locus 9q34.1
378: ADENOSINE MONOPHOSPHATE DEAMINASE 3; AMPD3
ERYTHROCYTE AMP DEAMINASE DEFICIENCY, INCLUDED
Gene map locus 11pter-p13
379: ADENOSINE MONOPHOSPHATE DEAMINASE 1; AMPD1
MYOADENYLATE DEAMINASE DEFICIENCY, MYOPATHY DUE TO, INCLUDED
Gene map locus 1p21-p13
380: ACTIN, ALPHA, SKELETAL MUSCLE 1; ACTA1
MYOPATHY, ACTIN, CONGENITAL, WITH CORES, INCLUDED
Gene map locus 1q42.1
381: ADENINE PHOSPHORIBOSYLTRANSFERASE; APRT
2,8-alphaDIHYDROXY ADENINE UROLITHIASIS, INCLUDED
Gene map locus 16q24.3
382: ACETYLCHOLINESTERASE; ACHE
YT BLOOD GROUP, INCLUDED
Gene map locus 7q22
383: ACETYL-CoA ACETYLTRANSFERASE 2; ACAT2
ACAT2 DEFICIENCY, INCLUDED
Gene map locus 6q25.3-q26
384: ALDEHYDE DEHYDROGENASE 2 FAMILY; ALDH2
ALCOHOL SENSITIVITY, INCLUDED
Gene map locus 12q24.2 [symbol]
indicates data missing or illegible when filed

TABLE 5
Inherited disorders

| | |
|---|---|
| 1. | monogenic disease, monogenic disorder - an inherited disease controlled by a single pair |
| | of genes |
| 2. | polygenic disease, polygenic disorder - an inherited disease controlled by several genes |
| | at once |
| 3. | achondroplasia, achondroplasty, chondrodystrophy, osteosclerosis congenita - an |
| | inherited skeletal disorder beginning before birth; cartilage is converted to bone resulting in |
| | dwarfism |
| 4. | abetalipoproteinemia - a rare inherited disorder of fat metabolism; characterized by |
| | severe deficiency of beta-lipoproteins and abnormal red blood cells (acanthocytes) and |
| | abnormally low cholesterol levels |
| 5. | inborn error of metabolism - any of a number of diseases in which an inherited defect |
| | (usually a missing or inadequate enzyme) results in an abnormality of metabolism |
| 6. | congenital megacolon, Hirschsprung's disease - congenital condition in which the colon |
| | does not have the normal network of nerves; there is little urge to defecate so the feces |
| | accumulate and cause megacolon |
| 7. | mucopolysaccharidosis - any of a group of genetic disorders involving a defect in the |
| | metabolism of mucopolysaccharides resulting in greater than normal levels of |
| | mucopolysaccharides in tissues |
| 8. | hyperbetalipoproteinemia - a genetic disorder characterized by high levels of beta- |
| | lipoproteins and cholesterol; can lead to atherosclerosis at an early age |
| 9. | ichthyosis - any of several congenital diseases in which the skin is dry and scaly like a |
| | fish |
| 10. | branched chain ketoaciduria, maple syrup urine disease - an inherited disorder of |
| | metabolism in which the urine has a odor characteristic of maple syrup; if untreated it can lead to |
| | mental retardation and death in early childhood |
| 11. | McArdle's disease - an inherited disease in which abnormal amounts of glycogen |
| | accumulate in skeletal muscle; results in weakness and cramping |
| 12. | dystrophy, muscular dystrophy - any of several hereditary diseases of the muscular |
| | system characterized by weakness and wasting of skeletal muscles |
| 13. | oligodactyly - congenital condition in which some fingers or toes are missing |
| 14. | oligodontia - congenital condition in which some of the teeth are missing |
| 15. | otosclerosis - hereditary disorder in which ossification of the labyrinth of the inner ear |
| | causes tinnitus and eventual deafness |
| 16. | autosomal dominant disease, autosomal dominant disorder - a disease caused by a |
| | dominant mutant gene on an autosome |
| 17. | autosomal recessive defect, autosomal recessive disease - a disease caused by the |
| | presence of two recessive mutant genes on an autosome |
| 18. | congenital pancytopenia, Fanconi's anaemia, Fanconi's anemia - a rare congenital anemia |
| | characterized by pancytopenia and hypoplasia of the bone marrow |
| 19. | juvenile amaurotic idiocy, Spielmeyer-Vogt disease - a congenital progressive disorder |
| | of lipid metabolism having an onset at age 5 and characterized by blindness and dementia and |
| | early death |
| 20. | congenital afibrinogenemia - a rare congenital disorder of blood coagulation in which no |
| | fibrinogen is found in the blood plasma |
| 21. | Albers-Schonberg disease, marble bones disease, osteopetrosis - an inherited disorder |
| | characterized by an increase in bone density; in severe forms the bone marrow cavity may be |
| | obliterated |
| 22. | nevoid elephantiasis, pachyderma - thickening of the skin (usually unilateral on an |
| | extremity) caused by congenital enlargement of lymph vessel and lymph vessel obstruction |
| 23. | dwarfism, nanism - a genetic abnormality resulting in short stature |
| | lactase deficiency, lactose intolerance, milk intolerance - congenital disorder consisting of an |
| | inability to digest milk and milk products; absence or deficiency of lactase results in an inability |
| | to hydrolyze lactose |
| 24. | porphyria - a genetic abnormality of metabolism causing abdominal pains and mental |
| | confusion |
| 25. | hepatolenticular degeneration, Wilson's disease - a rare inherited disorder of copper |
| | metabolism; copper accumulates in the liver and then in the red blood cells and brain |

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method comprising:
a) obtaining one or more tissue samples from a subject not previously diagnosed with a specific disorder;
b) obtaining one or more carboxy-terminal (C-terminal) antibodies to a target protein of a control sample wherein the one or more tissue samples and the control sample are of the same origin;
c) exposing the one or more tissue samples to the one or more C-terminal antibodies and allowing the one or more C-terminal antibodies to bind to the one or more tissue samples;
d) detecting the one or more C-terminal antibodies bound to the one or more tissue samples; and
e) assessing presence or risk of developing the specific disorder based on reduction or absence of bound one or more C-terminal antibodies to the target protein of the one or more tissue samples compared to bound one or more C-terminal antibodies to the target protein of the control sample.

2. (canceled)

3. The method of claim 1, wherein the specific disorder comprises an inherited genetic disorder.

4.-5. (canceled)

6. The method of claim 1, wherein the one or more tissue samples is selected from the group consisting of breast, prostate, bone marrow, ovarian, pancreatic, lung, brain, thyroid, bowel, skin and throat.

7. The method of claim 1, further comprising:
f) obtaining one or more amino-terminal (N-terminal) antibodies that bind to the target protein;
g) exposing the one or more tissue samples to the one or more N-terminal antibodies and allowing the one or more N-terminal antibodies to bind to the one or more tissue samples;
h) detecting the one or more N-terminal antibodies bound to the one or more tissue samples;
i) comparing the N-terminal bound antibodies to the C-terminal bound antibodies of the one or more tissue samples;
j) assessing presence or risk of a specific disorder in the subject based on the level of N-terminal bound antibodies to C-terminal bound antibodies of the one or more tissue samples wherein a decrease in C-terminal bound antibodies compared to N-terminal bound antibodies is indicative of an increase in risk of disease in the subject.

8. The method of claim 7, wherein the ratio of C-terminal bound antibodies to N-terminal bound antibodies is indicative of level of truncation of the target protein in the one or more tissue samples.

9. The method of claim 1, wherein detecting the bound one or more C-terminal antibodies is selected from the group consisting of detecting the bound one or more C-terminal antibodies using an immunohistochemistry (IHC) method, using Elisa analysis, using Western blot analysis, using immunoprecipitation, using GC-mass spectroscopy and a combination thereof

10. The method of claim 1, wherein the specific disorder comprises cancer, blood disease, immunological disease, infectious disease, endocrine disease, glandular disease, muscular disease, skeletal disease, skin disease, lung disease, gastrointestinal disease, heart disease, neurosensory disease, and seizure-related disease.

11. The method of claim 10, wherein the cancer comprises breast cancer, ovarian cancer, prostate cancer, pancreatic cancer, lung cancer, brain cancer, thyroid cancer, bowel cancer, stomach cancer, skin cancer or throat cancer or combination thereof

12. A method comprising:
a) obtaining one or more tissue samples from a subject not previously diagnosed with a hereditary disease;
b) obtaining one or more C-terminal antibodies to at least one of BRCA1 and BRCA2;
c) exposing the one or more tissue samples to the one or more C-terminal at least one of BRCA1 and BRCA2 antibodies and allowing the one or more C-terminal antibodies to bind to the one or more tissue samples;
d) detecting the one or more C-terminal antibodies bound to the one or more tissue samples; and
e) assessing presence or risk of the subject for developing the hereditary disease based on the reduction or absence of bound C-terminal at least one of BRCA1 and BRCA2 antibodies to the one or more tissue samples wherein a reduction or absence of bound antibody increases the risk of the subject developing the hereditary disease.

13. The method of claim12, wherein the hereditary disease comprises cancer.

14. The method of claim 13, wherein the cancer comprises breast, ovarian, prostate, pancreatic, or throat cancer.

15. The method of claim 12, further comprising:
f) obtaining one or more N-terminal antibodies to at least one of BRCA1 and BRCA2;
g) exposing the one or more tissue samples to the one or more N-terminal at least one of BRCA1 and BRCA2 antibodies and allowing the one or more antibodies to bind to the one or more tissue samples;
h) detecting the one or more N-terminal antibodies bound to the one or more tissue samples;
i) comparing the N-terminal bound at least one of BRCA1 and BRCA2 antibodies to the C-terminal bound at least one of BRCA1 and BRCA2 antibodies of the one or more tissue samples;
j) assessing presence or risk of the hereditary disease in the subject based on the level of N-terminal bound at least one of BRCA1 and BRCA2 antibodies to C-terminal bound at least one of BRCA1 and BRCA2 antibodies to the one or more tissue samples wherein a decrease in C-terminal bound at least one of BRCA1 and BRCA2 antibodies to N-terminal bound at least one of BRCA1 and BRCA2 antibodies is indicative of an increase in risk of disease or presence of disease in the subject and wherein the compared N-terminal and C-terminal antibodies are directed to the same protein.

16. The method of claim 12, wherein the hereditary disease is breast or ovarian cancer.

17.-18. (canceled)

19. A method comprising:
a) obtaining one or more tissue sample(s) from a subject not previously diagnosed with a specific disease;
b) obtaining one or more phosphospecific antibodies directed to a protein of a control sample wherein the one or more tissue samples and the control sample are of the same origin;
c) exposing the one or more tissue samples to the one or more phosphospecific antibodies and allowing the one or more phosphospecific antibodies to bind to the one or more tissue sample samples;
d) assessing presence or risk of the subject for developing the specific disease based on the level of bound one or more phosphospecific antibodies to the one or more tissue samples wherein a reduction or absence of bound antibody increases the risk of the subject developing the disease.

20. The method of claim 19, wherein the specific disease is sporadic or hereditary cancer.

21. The method of claim 20, wherein the sporadic or hereditary cancer comprises breast, ovarian, prostate, pancreatic, or throat cancer.

22. The method of claim 21, wherein the cancer is estrogen-positive sporadic breast cancer.

23. A method comprising:
a) obtaining one or more tissue samples from a subject not previously diagnosed with a specific cancer;
b) obtaining one or more phosphospecific antibodies directed to bind BRCA2;
c) exposing the one or more tissue samples to the one or more phosphospecific antibodies and allowing the one or more phosphospecific antibodies to bind to the one or more tissue samples;
d) predicting response of the subject to a therapeutic treatment based on level of bound phosphospecific antibodies to the one or more tissue samples.

24. The method of claim 23, wherein one of the one or more phosphospecific antibodies comprises serine 3291 (S3291) BRCA2 phosphospecific antibody.

25.-27. (canceled)

28. The method of claim 23, wherein a reduction in the level of bound BRCA2 phosphospecific bound antibody indicates an increase in sensitivity of the subject to a PARP inhibitor therapy.

29.-32. (canceled)

33. A composition comprising a phosphospecific antibody that binds to BRCA2.

34. The composition of claim 33, wherein said composition comprises 80% or more of SEQ ID NO:1.

35. The composition of claim 33, wherein said composition is further conjugated to an agent.

36. (canceled)

37. A kit comprising:
a) a serine 3291 (S3291) BRCA2 phosphospecific antibody; and
b) a suitable container.

38. The kit of claim 37, further comprising one or more C-terminal antibodies directed to bind BRCA2.

39. (canceled)
